SEQUENCE LISTING (37 sequences)

SEQ ID NO: 1 - DNA, 15 bp, Artificial Sequence
  Description of Artificial Sequence Adaptor
gtcctcgatgtgcgc

SEQ ID NO: 2 - DNA, 11 bp, Artificial Sequence
  Description of Artificial Sequence Adaptor
acatcgaggac

SEQ ID NO: 3 - DNA, 17 bp, Artificial Sequence
  Description of Artificial Sequence Primer
gtcctcgatgtgcgcwn

SEQ ID NO: 4 - DNA, 25 bp, Artificial Sequence
  Description of Artificial Sequence Adaptor
ccaaacccgcttattctccgcagta

SEQ ID NO: 5 - DNA, 29 bp, Artificial Sequence
  Description of Artificial Sequence Adaptor
nnnntactgcggagaataagcgggtttgg

SEQ ID NO: 6 - DNA, 25 bp, Artificial Sequence
  Description of Artificial Sequence Adaptor
gtgctctggtgctacgcatttaccg

SEQ ID NO: 7 - DNA, 29 bp, Artificial Sequence
  Description of Artificial Sequence Adaptor
nnnncggtaaatgcgtagcaccagagcac

SEQ ID NO: 8 - DNA, 25 bp, Artificial Sequence
  Description of Artificial Sequence Adaptor
ccgtggcaattagtcgtctaacgct

SEQ ID NO: 9 - DNA, 29 bp, Artificial Sequence
  Description of Artificial Sequence Adaptor
nnnnagcgttagacgactaattgccacgg

SEQ ID NO: 10 - DNA, 43 bp, Artificial Sequence
  Description of Artificial Sequence Primer
ctgggtaggtccgatttaggctttttttttttttttttttttv

SEQ ID NO: 11 - DNA, 21 bp, Artificial Sequence
  Description of Artificial Sequence Primer
ctgggtaggtccgatttaggc

SEQ ID NO: 12 - DNA, 14 bp, Artificial Sequence
  Description of Artificial Sequence Digested double-stranded DNA
cgcgaacgcgtacg

SEQ ID NO: 13 - DNA, 10 bp, Artificial Sequence
  Description of Artificial Sequence Digested double-stranded DNA
cgtacgcgtt

SEQ ID NO: 14 - DNA, 25 bp, Artificial Sequence
  Description of Artificial Sequence Adaptor
acgcatttaccgcgcgacgcgtacg

SEQ ID NO: 15 - DNA, 25 bp, Artificial Sequence
  Description of Artificial Sequence Adaptor
cgtacgcgtcgcgcggtaaatgcgt

SEQ ID NO: 16 - DNA, 30 bp, Artificial Sequence
  Description of Artificial Sequence Double-stranded product DNA
catcagatacgtagcgaaaaaaaaaaaaaa

SEQ ID NO: 17 - DNA, 32 bp, Artificial Sequence
  Description of Artificial Sequence Double-stranded product DNA
ttttttttttttttttcgctacgtatctgatg

SEQ ID NO: 18 - DNA, 18 bp, Artificial Sequence
  Description of Artificial Sequence Double-stranded product DNA
ttttttttttttttttcg

SEQ ID NO: 19 - DNA, 19 bp, Artificial Sequence
  Description of Artificial Sequence Double-stranded product DNA
acgcatttaccgcgcgacg

SEQ ID NO: 20 - DNA, 18 bp, Artificial Sequence
  Description of Artificial Sequence Digested double-stranded DNA
cgctacgcgtacggtagg

SEQ ID NO: 21 - DNA, 14 bp, Artificial Sequence
  Description of Artificial Sequence Digested double-stranded DNA
cctaccgtacgcgt

SEQ ID NO: 22 - DNA, 25 bp, Artificial Sequence
  Description of Artificial Sequence Adaptor
acgcatttaccgcgctacgcgtacg

SEQ ID NO: 23 - DNA, 25 bp, Artificial Sequence
  Description of Artificial Sequence Adaptor
cgtacgcgtagcgcggtaaatgcgt

SEQ ID NO: 24 - DNA, 17 bp, Artificial Sequence
  Description of Artificial Sequence Double-stranded product DNA
ttttttttttttttttc

SEQ ID NO: 25 - DNA, 12 bp, Artificial Sequence
  Description of Artificial Sequence Double-stranded product DNA
acgcatttaccg

SEQ ID NO: 26 - DNA, 20 bp, Artificial Sequence
  Description of Artificial Sequence Primer
aggacatttgtgagtcaggc

SEQ ID NO: 27 - DNA, 20 bp, Artificial Sequence
  Description of Artificial Sequence Primer
ttcacgctggactgtttcgg

SEQ ID NO: 28 - DNA, 40 bp, Artificial Sequence
  Description of Artificial Sequence Size marker
ctagtcctgcaggtttaaacgaattcgcccttggatgcct

SEQ ID NO: 29 - DNA, 40 bp, Artificial Sequence
  Description of Artificial Sequence Size marker
ctagaggcatccaagggcgaattcgtttaaacctgcagga

SEQ ID NO: 30 - DNA, 799 bp, Artificial Sequence
  Description of Artificial Sequence Internal control
aggacatttgtgagtcaggcgtgtcttggatgcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvaaaaaaaaaaaaaaaaaaaaaaaaaaaaaaaaaaaaaaaaaccgaaacagtccagcgtgaattgg

SEQ ID NO: 31 - DNA, 33 bp, Artificial Sequence
  Description of Artificial Sequence Adaptor
aggacatttgtgagtcaggcgtgtcttggatgc

SEQ ID NO: 32 - DNA, 37 bp, Artificial Sequence
  Description of Artificial Sequence Adaptor
nnnngcatccaagacacgcctgactcacaaatgtcct

SEQ ID NO: 33 - DNA, 64 bp, Artificial Sequence
  Description of Artificial Sequence Primer
ccaattcacgctggactgtttcggtttttttttttttttttttttttttttttttttttttttt

SEQ ID NO: 34 - DNA, 49 bp, Artificial Sequence
  Description of Artificial Sequence Primer
ccaattcacgctggactgtttcggttttttttttttttttttttttttt

SEQ ID NO: 35 - DNA, 13 bp, Artificial Sequence
  Description of Artificial Sequence Primer
gtgtcttggatgc

SEQ ID NO: 36 - DNA, 26 bp, Artificial Sequence
  Description of Artificial Sequence Primer
tttttttttttttttttttttttttv

SEQ ID NO: 37 - DNA, 43 bp, Artificial sequence
  Primer
ttttttttttttttttttttttttttttttttttttttttvnn

(19) United States
(12) Patent Application Publication
(10) Pub. No.: US 2003/0175908 A1
(43) Pub. Date: Sep. 18, 2003

(54) Methods and means for manipulating nucleic acid

(76) Inventors: Sten Linnarsson, Stockholm (SE); Patrik Ernfors, Stockholm (SE); Goran Bauren, Stockholm (SE); Ats Metsis, Stockholm (SE); Arno Pihlak, Stockholm (SE); Andreas Montelius, Stockholm (SE)

Correspondence Address: NIXON & VANDERHYE, PC, 1100 N GLEBE ROAD, 8TH FLOOR, ARLINGTON, VA, 22201-4714, US

(21) Appl. No.: 10/352,253

(22) Filed: Jan. 28, 2003

Related U.S. Application Data
(60) Provisional application No. 60/352,215, filed on Jan. 29, 2002.

(51) Int. Cl. C12P 19/34; C07H 21/00

(52) U.S. Cl. 435/91.51; 536/22.1; 435/91.52; 435/91.53

(57) ABSTRACT

Methods of manipulation of nucleic acid, in particular amplification by means of the polymerase chain reaction (PCR), including use of oligonucleotides and combinations and kits comprising such oligonucleotides, also methods comprising use of nested PCR, allowing for improved results in methods wherein large numbers of nucleic acid fragments are manipulated by means of PCR and electrophoresis. Oligonucleotides are provided for use a size standards in electrophoresis, and internal controls allowing for calculation of relative amounts of material present. Improved results can be achieved in methods of profiling mRNA transcribed in a system under investigation.

The present invention relates to manipulation of nucleic acid, in particular amplification by means of the polymerase chain reaction (PCR). More specifically, the invention relates to oligonucleotides and combinations and kits comprising such oligonucleotides, also methods comprising use of nested PCR. Embodiments of the present invention allow for improved results in methods wherein large numbers of nucleic acid fragments are manipulated by means of PCR and electrophoresis. The present invention further provides oligonucleotides for use a size standards in electrophoresis, and internal controls allowing for calculation of relative amounts of material present. The present invention allows for improved results in methods of profiling mRNA transcribed in a system under investigation.

Only a fraction of the total number of genes present in the genome is expressed in any given cell. The relatively small fraction of the total number of genes that is expressed in a cell determine its life processes e.g. intrinsic and extrinsic properties of the cell including development and differentiation, homeostasis, its response to insults, cell cycle regulation, aging, apoptosis, and the like.

Alterations in gene expression decide the course of normal cell development and the appearance of diseased states, such as cancer. Because the profile of gene expression in any given cell has direct consequences to its nature, methods for analyzing gene expression on a global scale are of critical import. Identification of gene-expression profiles will not only further understanding of normal biological processes in organisms but provide a key to prognosis and treatment of a variety of diseases or condition states in humans, animals and plants associated with alterations in gene expression. In addition, since differential gene expression is associated with predisposition to diseases, infectious agents and responsiveness to external treatments (Alizadeh et al., 2000; Cho et al., 1998; Der et al., 1998; Iyer et al., 1999; McCormick, 1999; Szallasi, 1998), identification of such gene-expression profiles can provide a powerful diagnostic tool for diseases, and as a tool, to identify new drugs for treating or preventing such diseases. This technology will also be immensely powerful for gene-discovery.

The only means of achieving this is to measure all genes expressed in particular tissues/cells at a particular time on a large scale, preferentially in one experiment. Less than a decade ago the concept of being able to simultaneously measure the concentration of every transcript in a cell in a single experiment would have been deemed undoable. However, use of DNA microarrays and other technological advances in the past few years have stimulated an extraordinary surge of interest in this field (Bowtell, 1999; Brown and Botstein, 1999; Duggan et al., 1999; Lander, 1999; Southern et al., 1999).

Microarrays have some disadvantages, but a number of alternative methods for detection and quantification of gene expression are available. These include for instance Northern blot analysis (Alwine et al., 1977), S1 nuclease protection assay (Berk and Sharp, 1977), serial analysis of gene expression (SAGE) (Velculescu et al., 1995) and sequencing of cDNA libraries (Okubo et al., 1992). However, all these are low-throughput approaches not suitable for global gene expression analysis. Differential display (Liang and Pardee, 1992) and related technologies contrast to microarray technology by not being based on solid support. The advantage of these technologies to microarrays is that no prior sequence information is required to execute the experiment. However, differential display and related technologies have two shortcomings that make them unsuitable for large-scale gene expression analysis; (i) the identity of the genes which are under study in.each experiment. can only be determined following cloning and sequence analysis of each of the cDNA in every experiment and (ii) the mRNAs are identified multiple times in every experiment.

A number of methods based on PCR have been proposed. A method for large scale restriction fragment length polymorphism of genomic DNA (KeyGene EP0969102) involves enzymatic cleavage of genomic DNA with one or two restriciton enzymes and ligating specific adapters to the fragments. Celera's GeneTag process is based on the principle that unique PCR fragments are generated for each cDNA. The fragments are separated by fluorescent capillary electrophoresis, then size-called and quantitated using Celera's proprietary algorithms. The amount of a specific mRNA is then determined by the fluorescent intensity of its cognate PCR fragment. Using Celera's proprietary GeneTag database, the cDNA fragment peaks are matched with their corresponding gene names. Another method (U.S. Pat. Nos. 6,010,850 and 5,712,126) uses a Y-shaped adaptor to suppress non-3′-fragments in the PCR. Thus, this cDNA is digested with a restriction enzyme and ligated to a Y-shaped adapter. The Y-shaped adapter enables selective amplification of 3′-fragments. Digital Gene Technologies (http://www.dgt.com or find DGT using any web browser) provide display of unique 3′-fragments, each representing a single gene and with each gene represented only once. The method (US patent 5459037) involves isolating and subcloning 3′-fragments, growing the subcloned fragments as a library in E. coli, extracting the plasmids, converting the inserts to CRNA and then back to DNA and then PCR amplifying.

We have previously described a PCR-based mRNA profiling method that allows direct identification of the expressed genes (GB0018016.6 and PCT/IB01/01539). In brief, cDNA generated from mRNA in a sample is subject to restriction enzyme digestion at one end, the other end being anchored to a solid support (such as beads, e.g. magnetic or plastic, or any other solid support that can be retained while washing, for instance by centrifugation or magnetism, or a microfabricated reaction chamber with sub-chambers for the subdivision procedure, where chemicals are washed through the chambers) by means of oligo T at the 5′ end of one strand—complementary to polyA originally at the 3′ end of the mRNA molecules. An adaptor is ligated to the free (digested) end of the cDNA molecules and PCR performed using primers that anneal at the ends of the cDNA—one designed to anneal to the adaptor at the 3′ end of one strand of the cDNA, the other containing oligodT to anneal to polyA at the 3′ end of the other strand of the cDNA (corresponding to the original polyA in the mRNA). For use with a Type II enzyme, each primer includes a variable nucleotide or sequence of nucleotides that will amplify a subset of cDNA's with complementary sequence—either adjacent to the adaptor for one strand or adjacent to the polyA for the other strand. For a Type IIS enzyme, adaptors are employed that will ligate with the possible different cohesive ends generated when the enzyme cuts the double-stranded DNA. Thus a population of adaptors may be employed to be complementary to all possible cohesive ends within the population of DNA after cutting/digestion by the Type IIS enzyme. Primers are used in the PCR that anneal with the adaptors.

Primers may be labelled, and the labels may correspond to the relevant A, T, C or G nucleotide at a corresponding position in the relevant primer variable region. This means that double-stranded DNA produced in the PCR is labelled, and that the combination of the label and the length of the product DNA provides a characteristic signal. Otherwise, the combination of length of the product and (i) PCR primer used for a Type II enzyme digest or (ii) adaptor used for a Type IIS digest, provides a characteristic signal.

From this, it should be understood that each gene gives rise to a single fragment and each complete profile thus shows each gene once; however, each fragment in a profile may correspond to multiple genes that happen to give rise to fragments of the same length occurring. in the same sub-reaction. This is the reason why simple database lookup is not sufficient to unambiguously identify most genes. By varying the enzyme used, multiple independent profiles can be generated, which allows more powerful combinatorial identification algorithms to be used (GB0018016.6 and PCT/IB01/01539).

It is clear that PCR-based methods give superior quantitative data with sensitivity and reproducibility that far exceed those of hybridisation-based methods, especially for samples amplified with a single primer pair.

The inventors have now established areas of improvement to increase reliability of quantitative data of any PCR-based RNA profiling method.

Aspects of the reactions where the inventors have identified relate to the following:

1. differential loading of the.subreaction onto capillaries for electrophoresis and other capillary-to-capillary effects;

2. differential loading of short and long fragments onto the capillaries because of competition between ions during electrokinetic injection;

3. sequence-dependent variations-in the apparent size of fragments in electrophoresis when judged against a size standard, especially when the size standard is qualitatively different in sequence composition from the fragments being judged;

4. differential amplification efficiencies for fragments of different length and/or sequence composition caused by the properties of the DNA polymerase used;

5. background non-specific fragments arising during PCR.

The aim is to obtain reliable quantitative information from the concurrent amplification of hundreds of fragments in a single reaction tube. Although all fragments in each reaction are amplified with a single primer pair and thus nominally with the same efficiency, differences may still arise because the DNA polymerase has a tendency to fall off longer fragments during elongation. This can result in a drop in amplification efficiency which is enzyme-dependent (i.e. enzymes from different species or different manufacturers have specific efficiency curves). Additionally, there are sequence composition-dependent differences in amplification efficiency. Compounding these effects is the effect of differential injection arising due to the way capillary electrophoresis is performed, where longer fragments tend to be less efficiently loaded onto the capillaries.

The present invention relates to primers and internal controls that may be used to reduce quantitative errors in PCR-based RNA profiling.

### BRIEF DESCRIPITON OF THE FIGURES

FIG. 1 outlines an approach to production of a single pattern characteristic of a sample, employing a Type II restriction enzyme (HaeII).

FIG. 2 outlines an alternative approach to production of a single pattern characteristic of a sample, employing a Type IIS restriction enzyme (FokI).

FIG. 3 shows the results of an experiment assessing specificity of ligation for an adaptor blocked on one strand. A single template oligonucleotide was used, having a four base pair single-stranded overhang, and adaptors were designed having a single stranded region exactly complementary to this, or with 1, 2 or 3 mismatches. Adaptors were ligated to the template oligonucleotide, and the products were amplified using PCR.

FIG. 4 outlines an embodiment of the method for generating a full profile for the mRNA molecules present in a sample, using a combinatorial algorithm of the invention. Steps I to VII are shown.

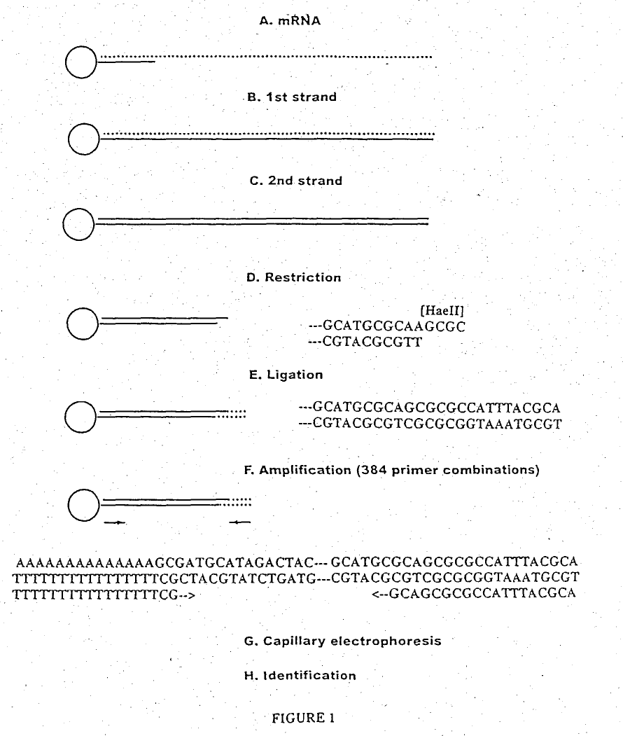

In step I, mRNA is captured on magnetic beads carrying an oligo- dT tail.

In step II, a complementary DNA strand is synthesized, still attached to the beads.

In step III, the mRNA is removed, and a second cDNA strand is synthesized. The double-stranded cDNA remains covalently attached to the beads.

In step IV, the double-stranded cDNA is split into two separate pools. Each pool is digested with a different restriction enzyme. The sequence of cDNA corresponding to the 3′ end of the mRNA remains attached to the beads.

In step V, adaptors are ligated to the digested end of the cDNA. In this embodiment of the invention, 256 different adaptors are ligated in 256 separate reactions. Also in this embodiment of the invention, the adaptors are blocked on one strand, so that PCR proceeds only from the other strand.

In step VI, each of the fractions is amplified with a single PCR primer pair.

In step VII, the PCR products are subject to capillary electrophoresis. This produces a independent pattern for each of the pools, digested by each of the restriction enzymes. These patterns can then be compared using a combinatorial algorithm of the invention, to identify the genes expressed in the sample.

FIG. 5 illustrates use of the size standard in accordance with an embodiment of the present invention. Lower panel shows the size standard going from 10 bp to 1010 bp. The upper panel shows a standard curve obtained by plotting the retention time (time to reach detector; Y axis) versus the known fragment size (X axis). The middle panel shows the residuals when the size standard is fitted-numerically to the equation indicated in the upper panel. In contrast to commercially available size standards, the sizing error stays below +/−1 bp across the entire range.

FIG. 6 shows an overview of a nested PCR system in accordance with an embodiment of the present invention. The template comprises a cDNA fragment captured on a solid support (illustrated as a bead) by means of binding of a polyA adaptor to its polyA tail, and an adaptor sequence that anneals at the end distal to the polyA tail, for instance where the fragment has been digested using a Type II or Type IIS restriction enzyme (e.g. as discussed further elsewhere herein). Only one template is shown, but the invention is generally concerned with amplification of populations of fragments generated by digestion of multiple fragments (e.g. cDNA copies of total mRNA present in a sample). In a nested PCR, there is a first round of PCR (PCR#1) where primers anneal to the adaptors at each end (forward primer shown to the left of the figure and the back primer shown to the right of the figure), then a second round of PCR (PCR#2) where multiple primers are used to amplify the different templates in a population. Forward primers shown to the left anneal to a variable part of the adaptor and extend into the sequence of digested CDNA fragment, while the back primers anneal to junction with the polyA tail. Back primers are shown in the figure as labelled, each of three possible back primers—with A, G or C as the 3′ nucleotide shown to the left of the back primer (the remainder being oligoT) - is labelled with a different label. (The A, G or C is complementary to the T, C or G residue immediately before the polyA sequence in the upper strand, corresponding to the polyA tail in the original mRNA). The product is, for each initial template cDNA fragment, of a defined length that represents the distance from the polyA tail to the site of adaptor annealing, itself where the restriction enzyme used in the digest actually cut the cDNA.

In FIG. 7, the left panel shows the result of amplifying a simple template (a double-stranded DNA molecule carrying the appropriate template sequences) using the different primer pairs indicated (primers A, B, C, D, E and F as disclosed elsewhere herein; Sz—size marker). Primer pair E/F clearly gives superior yield and shows no primer-dimer effects such as those shown by C/E. The right panel shows amplification of a simple target in the presence of a complex mix of DNA not carrying the template sequence. Again, primer pair E/F clearly is the most specific, showing only a faint band below the specific target band, in contrast with the smear shown by primers A/B. Primer A has sequence SEQ ID NO. 4; primer B has sequence SEQ ID NO. 11, primer E has sequence 5′-AGGACATTTGTGAGTCAGGC-3′ (SEQ ID NO. 26); primer F has sequence 5′-TTCACGCTGGACTGTTTCGG-3′ (SEQ ID NO. 27).

FIG. 8 shows a portion of a signal obtained by capillary electrophoresis. Each peak in the diagram corresponds to a fragment in the original sample. Time (the horizontal axis) corresponds to fragment length because longer fragments are delayed during electrophoresis by a polymer in the capillary. The vertical axis corresponds to fluorescence signal intensity and shows the abundance of each fragment class in the original sample. The magnified portion shows the unusually high reproducibility where two independent reactions performed on the same sample show almost indistinguishable peak patterns.

FIG. 9 shows the same experiment as FIG. 8, except that ligase was omitted when ligating adaptor in the reaction shown in the lighter grey. The almost complete lack of PCR background is evident, and it is notable that the total amount of background signal contributes less than 0.1% of the total signal.

Primers for use in nested PCR in accordance with the present invention are useful in amplifying DNA fragments, wherein one strand of the DNA fragment corresponds to a fragment of mRNA comprising a polyA tail. Such amplification is useful in a variety of contexts, including but not limited to embodiments of RNA profiling and fingerprinting as discussed further herein, with reference also to GB0018016.6 and PCT/IB0l/0539.

In accordance with one aspect of the present invention there is provided a method of providing a population of double-stranded product DNA molecules, the method comprising:

annealing polyA tails of mRNA molecules in a sample to an oligoT adaptor, which oligoT adaptor comprises a 3′ oligoT portion and a 5′ first back primer annealing sequence,

synthesizing a cDNA strand complementary to the mRNA molecules using the mRNA molecules as template, thereby providing a population of first cDNA strands;

removing the mRNA;

synthesizing a second cDNA strand complementary to each first strand, thereby providing a population of double-stranded cDNA molecules;

digesting the double-stranded cDNA molecules with a Type II or Type IIS restriction enzyme to provide a population of digested double-stranded cDNA molecules, each digested double-stranded cDNA molecule having a cohesive end provided by the restriction enzyme digestion;

ligating a population of cohesive adaptor oligonucleotides to the cohesive end of each of the digested double-stranded cDNA molecules, the cohesive adaptor oligonucleotides each comprising an end sequence complementary to a cohesive end, a first forward primer annealing sequence, and a second forward primer annealing sequence between the first forward primer annealing sequence and the cohesive end, thereby providing double-stranded template cDNA molecules each comprising a first strand and a second strand wherein the first strand of the double-stranded template cDNA molecules each comprise a 3′ terminal cohesive adaptor oligonucleotide and the second strand of the double-stranded template cDNA molecules each comprise a 3′ sequence complementary to the oligoT adaptor sequence;

purifying said double-stranded template cDNA molecules;

performing a first polymerase chain reaction on the double-stranded template cDNA molecules having a sequence complementary to a 3′ end of an mRNA using a first forward primer, which comprises a sequence which anneals to the first forward primer annealing sequence, and a first back primer, which comprises a sequence which anneals to the first back primer annealing sequence;

performing a second polymerase chain reaction amplification on products of the first polymerase chain reaction using a population of second forward primers and a population of second back primers,

wherein the second forward primers each comprise a sequence which anneals to a second forward primer annealing sequence of a cohesive adaptor oligonucleotide; and

where the restriction enzyme is a Type II enzyme the second forward primers each comprise at least one 3′ terminal variable nucleotide and optionally more than one 3′ terminal variable nucleotides wherein the variable nucleotide is, or at a corresponding position within the variable nucleotides each second forward primer has, a nucleotide selected from A, T, C and G, whereby the population of second forward primers primes synthesis in the polymerase chain reaction of first strand product DNA molecules each of which is complementary to the first strand of a template cDNA molecule that comprises adjacent to the primer annealing sequence within the first strand of the template cDNA molecule a nucleotide or sequence of nucleotides complementary to the variable nucleotide or nucleotides of a second forward primer within the population of second forward primers; or

where the restriction enzyme is a Type IIS enzyme the second forward primers prime synthesis in the polymerase chain reaction of first strand product DNA molecules each of which is complementary to the first strand of a template cDNA molecule that comprises within the first strand of the template cDNA molecule a sequence of nucleotides complementary to an end sequence of a cohesive adaptor oligonucleotide in the population of cohesive adaptor oligonucleotides;

the second back primers comprise an oligot sequence and a 3′ variable portion conforming to the following formula: (G/C/A) (X)ₙ wherein X is any nucleotide, n is zero, at least one or more than one; whereby the population of second back primers primes synthesis in the polymerase chain reaction of second strand product DNA molecules each of which is complementary to the second strand of a template cDNA molecule that comprises adjacent to polyA within the second strand of the template cDNA molecule a nucleotide or nucleotides complementary to the variable portion of a second back primer within the population of second back primers;

whereby performing the polymerase chain reaction amplifications provides a population of double-stranded product DNA molecules each of which comprises a first strand product DNA molecule and a second strand product DNA molecule.

Removing mRNA from the first strand may be by any approach available in the art. This may involve for example digestion with an RNase, which may be partial digestion, and/or displacement of the mRNA by the DNA polymerase synthesizing the second cDNA strand (as for example in the Clontech™ SMART™ system).

The method may further comprise separating double-stranded product DNA molecules on the basis of length; and

detecting said double-stranded product DNA molecules;

whereby a pattern for the population. of mRNA molecules present in the sample is provided by combination of length of said double-stranded product DNA molecules and (i) second forward primer variable nucleotide or nucleotides, where a Type II restriction enzyme is employed, or (ii) cohesive adaptor oligonucleotide end sequence, where a Type IIS restriction enzyme is employed.

A method according to further embodiments of the present invention may further comprise:

generating an additional pattern for the sample using a second, different Type II or Type IIS restriction enzyme, and comparing the patterns generated using at least two different Type II or Type IIS restriction enzymes in separate experiments with a database of signals determined or predicted for known mRNA's.

Patterns may be generated using at least two different Type II or Type IIS.restriction enzymes in separate experiments with a database of signals determined or predicted for known mRNA's by:

(i) listing all mRNA's in the database which may correspond to a double-stranded product DNA in each experiment, forming a list of mRNA molecules possibly present in the sample for each experiment, and

(ii) for each experiment listing mRNA's which definitely do not correspond to a double-stranded product DNA molecule, forming a list of mRNA molecules definitely not present in the sample for each experiment, then

(iii) removing the mRNA molecules definitely not present in the sample from the list of mRNA molecules possibly present for each experiment, and

(iv) generating a list of mRNA molecules possibly present in the sample and mRNA molecules definitely not present in the sample by combining each list generated for each experiment in (iii);

thereby providing a profile of mRNA molecules present in the sample.

Patterns generated using at least two different Type II or Type IIS restriction enzymes in separate experiments may be compared with a database of signals determined or predicted for known mRNA's, by:

(i) listing all mRNA's in the database which may correspond to a double-stranded product DNA in each experiment, and forming a set of equations of the form Fi=m₁+m₂+m₃, wherein Fi is the intensity of the signal from the fragment, the numerals are the mRNA identity and wherein each mRNA which may correspond to a double-stranded product DNA appears as a term on the right-hand side;

(ii) for each experiment listing mRNA's which definitely do not correspond to double-stranded product DNA in each experiment, and writing for each gene which definitely does not correspond to a double-stranded product DNA in each experiment an equation of the form 0=m₄, wherein the numeral is the mRNA identity;

(iii) combining the sets of equations to form a system of simultaneous equations wherein the number of equations is greater than the number of genes in the organism;

(iv) determining an estimate of the expression level of each gene by solving the system of simultaneous equations, thereby providing a profile of mRNA molecules present in the sample.

The following primers may be employed:

first forward primer of the following sequence:

5′-AGGACATTTGTGAGTCAGGC-3′ (SEQ ID NO. 26),

first back primer of the following sequence:

5′-TTCACGCTGGACTGTTTCGG-3′ (SEQ ID NO. 27),

second forward primer of the following sequence:

5′-GTGTCTTGGATGC-3′ (SEQ ID NO. 35), and

second back primer of the following sequence:

5′-(T)_{z}VN₁N₂, wherein z is 10-40, V is A, G or C, N₁ is optional and if present is A, G, C or T, and N₂ is optional and if present is A, G, C or T.

Where z is between 10 and 40, this provides an oligoT run wherein there are 10 to 40 T's. Preferably there are 15-30, and there may be 15, 16, 17, 18, 19, 20, 21, 22, 23, 24, 25, 26, 27, 28, 29 or 30. More preferably there are about 25.

In a further aspect, the present invention provides a method of amplifying cDNA fragments to provide a population of double-stranded product DNA molecules, each cDNA fragment comprising an upper strand that comprises a copy of a 3′ fragment of an mRNA molecule comprising a polyA tail, and a lower strand that is complementary to the upper strand, wherein the upper strand comprises at its 5′ terminus the following adaptor (1) sequence:

5′-AGGACATTTGTGAGTCAGGCGTGTCTTGGATGC-3′, and the lower strand comprises at its 3′ terminus the following adaptor (2) sequence:

5′-p (N)ₓGCATCCAAGACACGCCTGACTCACAAATGTCCT-3′, and wherein the lower strand comprises at its 5′ terminus the following adaptor (3) sequence:

5′-CCAATTCACGCTGGACTGTTTCGG-(T)_{y}-3′ and the upper strand comprises at its 3′ terminus the following adaptor (4) sequence:

5′-(A)_{y}-CCGAAACAGTCCAGCGTGAATTGG-3′,

wherein the upper and lower strands are provided by ligation of adaptors of adaptor sequence (1) and (2) following restriction digest of cDNA fragments, wherein N is A, T, C or G, and wherein x corresponds to the number of bases of overhang created by the restriction digest;

the method comprising performing nested polymerase chain reaction,

wherein a first polymerase chain reaction is performed with a first forward primer of the following sequence:

5′-AGGACATTTGTGAGTCAGGC-3′ (SEQ ID NO. 26), and a first back primer of the following sequence:

5′-TTCACGCTGGACTGTTTCGG-3′ (SEQ ID NO. 27), and

wherein a second polymerase chain reaction is performed with a second forward primer of the following sequence:

5′-GTGTCTTGGATGC-3′ (SEQ ID NO. 35), and a second back primer of the following sequence:

5′-(T) VN₁N₂, wherein z is 10-40, V is A, G or C, N, is optional and if present is A, G, C or T, and N₂ is optional and if present is A, G, C or T.

The second back primers may be labelled, e.g. with fluorescent dyes readable by a sequencing machine.

Double-stranded CDNA may be generated from mRNA in a sample. This double-stranded cDNA may be subject to restriction enzyme digestion to provide digested double-stranded cDNA molecules, each having a cohesive end provided by the restriction enzyme digestion.

A population of adaptors may be ligated to the cohesive ends of each of the digested double-stranded cDNA molecules, thereby providing double-stranded template cDNA molecules each comprising a first strand and a second strand, wherein the first strand of the double-stranded template cDNA molecules each comprise a 3′ terminal adaptor oligonucleotide and the second strand of the double-stranded template cDNA molecules each comprise a 3′ terminal polyA sequence.

These double-stranded template cDNA molecules can then be purified. There is thus provided a substantially pure population of cDNA fragments having a sequence complementary to a 3′ end of an mRNA.

Purification of the double-stranded template cDNA molecules may be achieved by any suitable means available to the skilled person. For example, the polyA or polyT sequence at one end of the cDNA molecule may be tagged with biotin, allowing purification of these double-stranded template cDNA molecules by binding to streptavadin-coated beads. Alternatively, isolation of these double-stranded template cDNA molecules may be achieved by hybridisation selection, dependent on binding to an oligoT and/or oligoA probe, prior to PCR.

Preferably, digested double-stranded CDNA comprising a strand having a 3′ terminal polyA sequence, are purified prior to ligating the adaptor oligonucleotides. This has the advantage of preventing non-specific ligation of adaptors. Again, this may employ any of the methods available to the skilled person, including purification by biotin tagging, as described above.

The 3′ ends of the cDNA sequence may be immobilised prior to restriction digestion. In this embodiment, one end of the cDNA generated from the mRNA is anchored to a solid support (such as beads, e.g. magnetic or plastic, or any other solid support that can be retained while washing, for instance by centrifugation or magnetism, or a microfabricated reaction chamber with sub-chambers for the subdivision procedure, where chemicals are washed through the chambers) by means of oligoT at the 5′ end—complementary to polyA originally at the 3′ end of the mRNA molecules. The other end of the cDNA sequence is subject to restriction enzyme digestion, and an adaptor is ligated to the free (digested) end. Purification of the above described digested double-stranded cDNA molecules or double-stranded template cDNA molecules may thus be achieved by washing away excess materials, while retaining the desired molecules on the solid support.

PCR is performed using primers that anneal at the ends of the cDNA—one designed to anneal to the adaptor at the 3′ end of one strand of the cDNA, the other containing oligodT to anneal to polyA at the 3′ end of the other strand of the cDNA (corresponding to the original polyA in the mRNA). For use with a Type II enzyme, each primer includes a variable nucleotide or sequence of nucleotides that will amplify a subset of cDNA's with complementary sequence—either adjacent to the adaptor for one strand or adjacent to the polyA for the other strand. For a Type IIS enzyme, adaptors are employed that will ligate with the possible different cohesive ends generated when the enzyme cuts the double-stranded DNA. Thus a population of adaptors may be employed to be complementary to all possible cohesive ends within the population of DNA after cutting/digestion by the Type IIS enzyme. Primers are used in the PCR that anneal with the adaptors.

Primers may be labelled, and the labels may correspond to the relevant A, T, C or G nucleotide at a corresponding position in the relevant primer variable region. This means that double-stranded DNA produced in the PCR is labelled, and that the combination of the label and the length of the product DNA provides a characteristic signal. Otherwise, the combination of length of the product and (i) PCR primer used for a Type II enzyme digest or (ii) adaptor used for a Type IIS digest, provides a characteristic signal.

Thus, where the present invention is used in a profiling context, each gene (mRNA in the sample) gives rise to a single fragment and each complete pattern thus shows each gene once. The pattern may be characteristic of the sample.

A pattern of signals generated for a sample, or one or more individual signals identified as differing between samples, may be compared with a pattern generated from a database of known sequences to identify sequences of interest.

Patterns generated from different cells or the same cells under different conditions or stages of differentiation or cell cycle, or transformed (tumorigenic) cells and normal cells, can be compared and differences in the pattern identified. This allows for identification of sequences whose expression is involved in cellular processes that differ between cells or in the same cells under different conditions or stages of differentiation or cell cycle or between normal and tumorigenic cells.

However, each fragment in a pattern may correspond to multiple genes that happen to give rise to fragments of the same length occurring in the same sub-reaction. These multiple genes, which will appear as doublets during analysis, cannot be distinguished by a simple database look-up.

In order to increase the number of genes which can be unambiguously identified by the procedure, a second, independent pattern may be obtained using a different restriction enzyme. This allows the patterns to be compared to a database of signals determined or predicted for known mRNAs using a combinatorial identification algorithm. This greatly increases the number of genes which can be unambiguously identified, for reasons discussed under the section “fragment identification”.

The combinatorial algorithm can be performed by a computer as follows:

1. All the genes in the database which correspond to a fragment in each experiment are listed. This forms a list of possibly expressed genes for each experiment.

2. Then for each experiment, the genes which definitely do not correspond to a fragment are listed (i.e. those which should give a fragment of a length which was not found in the experiment). This forms a list of definitely unexpressed genes for each experiment.

3. The unexpressed genes in each experiment are then removed from the list of possibly expressed genes in each other experiment.

4. The result is a list for each experiment where in most cases each fragment retains a single candidate gene identification.

A preferred algorithm allows both identification and quantification of the fragments. This embodiment may be especially suitable when all or most genes in an organism have been identified, and can be performed as follows:

1. All the genes in the database which correspond to a fragment in each experiment are listed. This forms a list of possibly expressed genes for each experiment. For each fragment in each experiment an equation is written of the form Fi=m₁+m₂+m₃, where 1, 2, 3 etc are the id's of the genes and Fi is the intensity of the signal from the fragment. Each gene which may correspond to a fragment peak in the electrophoresis appears as a term on the right-hand side.

For example, if a peak at 162 bp corresponds to genes 234, 647 and 78 in the database, and it has intensity 2546, then the corresponding equation is written:

2546=m₂₃₄+m₆₄₇+m₇₈

2. Then for each experiment, the genes which definitely do not correspond to a fragment are listed (i.e. those which should give a fragment of a length whith was not found in the experiment). This forms a list of definitely unexpressed genes for each experiment. For each gene on that list, an equation is written of the form:

0=m₆₅₇

Where 657 is the gene id, as above.

3. A system of simultaneous equations is thus obtained with m (=the number of genes in the organism) unknowns and n km equations (where k is the number of experiments). If all genes run as singlets in all experiments then n=km because each gene will appear in its own equation. The more they run as doublets or multiplets the smaller n-will be. As long as n>m, however, the system is over-determined and can thus be solved using standard numerical methods to find a least-squares solution. For example, the backslash operator in MATLAB can be used.

4. The solution of the system gives for each gene the best approximation of its expression level. The solution may be the least-squares solution. The more experiments that are performed, the better the approximation will be. Errors can be estimated by computing residuals (that is, by inserting the estimated gene activities in the equations to obtain calculated peak intensities and comparing those to the measured intensities). Simulations show that a system of 100 000 equations in 50 000 unknowns can be solved in 16 hours on a regular PC.

The algorithm will produce a profile of the mRNAs present in a sample. The profiles for two different cell types or the same cells type under different conditions or different stages of the cell cycle may be compared. This allows identification of the sequences which are differentially expressed in the two cell types. Furthermore, quantitative as well as qualitative differences in expression may be identified.

For use in an embodiment of a profiling method of the invention as disclosed herein, a restriction enzyme is generally selected such that one obtains a size distribution which can be readily separated and length-determined with the fragment analysis method employed. The distribution of isolated 3′ end fragments obtained by cutting with a restriction enzyme is proportional to 1/x where x is the length. The scale of the distribution depends on the probability of cutting. If an enzyme cuts once in 4096 (six base pair recognition sequence), the distribution will extend too far for current capillary electrophoresis methods. 1/1024 or 1/512 is preferred. HaeII cuts 1/1024 because of its degenerate recognition motif. FokI cuts 1/512 because it recognizes five base pairs in either forward or reverse directions. A 4 bp-cutter cuts 1/256, which creates a too compressed distribution where doublets are more likely to occur. Thus enzymes like HaeII and FokI are preferred.

Thus a restriction enzyme employed in preferred embodiments may cut double-stranded DNA with a frequency of cutting of 1/256-1/4096 bp, preferably 1/512 or 1/1024 bp.

Where the restriction enzyme is a Type II restriction enzyme, it is preferred to use HaeII, ApoI, XhoII or Hsp 921. Where the restriction enzyme is a Type IIS restriction enzyme, it is preferred to use FokI, BbvI or Alw261. Other suitable enzymes are identified by REBASE (rebase.neb.com).

Preferably, the restriction enzyme digests double-stranded DNA to provide a cohesive end of 2-4 nucleotides. For a Type IIS restriction enzyme a cohesive end of 4 nucleotides is preferred.

As discussed, more information can be obtained by generating an additional pattern for the sample using a second, or second and third, different Type II or Type IIS restriction enzyme or enzymes.

In forward primers used for PCR following digestion with a Type II enzyme, there may be a single variable nucleotide, or a variable nucleotide sequence of more than one nucleotide, e.g. two or three. At each position in a variable sequence, forward primers may be provided such that each of A, C, G and T is represented in the population.

In back primers (comprising oligo dT), n may be 0, 1 or 2.

No variable nucleotide is need in the primers used for PCR where a Type IIS restriction enzyme is employed because variability in the adaptor sequence is provided by the cohesive end. Generally, where a Type IIS restriction enzyme is employed a population of. adaptors is provided such that all possible cohesive ends for the restriction enzyme are represented in the population, and each adaptor may be ligated to a fraction of the sample in a separate reaction vessel. The adaptor used in each reaction vessel will then be known and combination of this information with the length of double-stranded product DNA molecules provides the desired characteristic pattern.

In a preferred embodiment, when ligating adaptors, the adaptors may be blocked on one strand, e.g., chemically. This may be achieved using a blocking group such as a 3′ deoxy oligonucleotide, or a 5′ oligonucleotide in which the phosphate group has been replace by nitrogen, hydroxyl or another blocking moiety. This allows ligation at the other, unblocked strand and can be used to improve specificity. A specificity greater than 250:1 can be obtained. PCR can proceed from the single ligated strand. In addition, ligation conditions have been identified which improve ligation specificity and/or efficiency, as described in the materials and methods. It has been found that these conditions are advantageous in achieving specificity in the ligation of adaptors with up to four variable base pairs.

For convenience, multiple adaptors may be combined in a single reaction vessel, in which case each different adaptor in a given vessel (with a different end sequence complementary to a cohesive end within the population of possible cohesive ends provided by the Type IIS restriction enzyme digestion) comprises a different primer annealing sequence. For instance three different adaptors may be combined in one reaction vessel. Corresponding first primers are then employed, and these may be labelled to distinguish between products arising from the respective different adaptor oligonucleotides.

Where a Type II enzyme is used, the forward primers may be labelled, although where individual polymerase chain reaction amplifications are performed in separate reaction vessels there is already knowledge of which forward primer is used. Otherwise, labelling provides convenient information on which forward primer sequence is providing which double-stranded DNA product molecule.

Conveniently, three different forward primer PCR amplifications can be performed in each reaction vessel, with each forward primer being labelled appropriately (optionally with employment of a labelled size marker).

Separation may employ capillary or gel electrophoresis. A single label may be employed per reaction, with four dyes per capillary or lane, one of which may carry a size marker.

Thus, a pattern characteristic of a population of mRNAs in a first sample is obtained.

In a further aspect of the present invention, a size marker is provided, as discussed further elsewhere herein. Such a size marker is useful in electrophoresis, and especially in a profiling method for determining the length of gene fragments, which length may be used as a component part of the characteristic signal for each of a population of gene fragments as discussed.

In a further aspect of the present invention an internal control is provided, as discussed further elsewhere herein. When loading nucleic acid for electrophoresis to determine fragment length, the internal control may be used to compensate for differentials in loading efficiencies, when relative amounts of each fragment. amplified in a population are used as a component part of the characteristic signal for each of the population of gene fragments as discussed.

As discussed elsewhere, a first pattern characteristic of a population of mRNA molecules present in a first sample may be compared with a second pattern characteristic of a population of mRNA molecules present in a second sample. A difference may be identified between said first pattern and said second pattern, and a nucleic acid whose expression leads to the difference between said first pattern and said second pattern may be identified and/or obtained.

As a supplement or alternative, a signal provided for a double-stranded product DNA by combination of its length and first primer or adaptor oligonucleotide used may be compared with a database of signals for known expressed mRNA's. A known expressed mRNA in the sample may be identified.

The protocol can then repeated using a different restriction enzyme, so as to obtain a second, independent pattern for the first sample. The patterns generated by at least two different Type II or Type IIS restriction enzymes in different experiments are compared with a database of signals determined or predicted for known mRNAs, by means of the algorithm described above, thus providing more powerful fragment identification. The resultant profile can then be compared to the profile of a sample from a different cell type or from the same cell type under different conditions or at a different stage of differentiation, so as to identify quantitative or qualitative differences in the sequences expressed by the two cell populations.

Precautions and optimising steps can be taken by the ordinary skilled person in accordance with common practice.

Labels may conveniently be fluorescent dyes, allowing for the relevant signals (e.g. on a gel) following electrophoresis to separate double-stranded product DNA molecules on the basis of their length to be read using a normal sequencing machine.

A library of 3′ end cDNA fragments can be prepared on a solid support, where each transcript is represented by a unique fragment. The library can be displayed on a capillary electrophoresis machine after PCR amplification with fluorescent primers. In order to reduce the number of bands in each electropherogram, the initial library may be subdivided, e.g. using one of the following two methods (α) and (β).

(α) For libraries generated with an ordinary Type II enzyme, an adapter is ligated to the cohesive end of each fragment. The adaptor comprises a portion complementary to the cohesive end generated by the restriction enzyme and a portion to which a primer anneals. One primer annealing sequence may be used, or a small number, e.g. 2 or 3, of different sequences showing minimal cross-hybridisation, to allow that small number of independent reactions to proceed in a single reaction vessel. The library is then split into a number of different reaction vessels and a subset of the fragments in each vessel is PCR amplified using primers compatible with the 3′ (oligo-T) and 5′ (universal adapter) ends carrying a few extra bases protruding into unknown sequence. Thus in each reaction a different combination of protruding bases causes selective amplification of a subset of the fragments.

(β) For libraries generated by Type IIS enzymes—which cleave outside their recognition sequence giving a gene-specific cohesive end—the library is split into a number of different reaction vessels. A set of adapters is designed containing a universal invariant part and a variable cohesive end such that all possible cohesive ends are represented in the set. In each reaction vessel a single such adapter is ligated. The subset of fragments in each vessel carrying adapters is then amplified with universal high-stringency primers.

In both methods, the resulting reactions may be run separately on a capillary electrophoresis machine which quantifies the fragment length and abundance, indicating the relative abundances of the corresponding mRNAs in the original sample.

For each fragment, the following are known:

the restriction enzyme site used to generate (e.g. 4-8 bases);

its length;

sub-reaction (given by the subdivision method, but generally corresponding to an additional 4-6 bases). If the subdivision is done judiciously, enough information is generated to identify each fragment with known sequences from a database This may be performed by selecting a combination of fragment length distribution (given by the enzyme) and subdivision (given by the protruding bases and/or by the cohesive end (Type IIS)). As few as two bases (16 sub-reactions) or as many as 8 (65536 sub-reactions) can be used; if a small genome is being analyzed, a small number of sub-reactions may be enough; if a high-throughput analysis method is available a large number of sub-reaction allows the separation of very large numbers of genes. In practice, between four and six bases are usually used.

As noted, primers for use in nested PCR are provided as embodiments of the present invention.

The present invention also provides in a further aspect an oligonucleotide useful as a size marker in electrophoresis. As is discussed further below in the experimental section, the size marker of the invention can be used to achieve a resolution of length determination of <1 bp.

In accordance with a further aspect of the present.invention there is provided a size standard that comprises tandemly ligated oligonucleotides of the following sequences:

(SEQ ID NO. 28)

5′-CTAGTCCTGCAGGTTTAAACGAATTCGCCCTTGGATGCCT-3′,
and

(SEQ ID NO. 29)

3′-AGGACGTCCAAATTTGCTTAAGCGGGAACCTACGGAGATC-5′;

wherein the tandemly ligated oligonucleotides are amplifiable from vectors wherein the tandemly ligated oligonucleotides are inserted between an upstream primer binding site and a downstream oligoA sequence.

Further provided is a population of vectors, wherein vectors in the population comprise tandemly ligated oligonucleotides of between 0 and 25 repeats, amplification using said a primer that binds said upstream primer binding site and a primer that binds said oligoA providing a population of size marker oligonucleotides of different lengths.

Further provided is a vector or recombinant vector in which the size marker is included and from which the size marker may be excised, e.g. by restriction enzyme digest or from which the size marker can be amplified by means of polymerase chain reaction (PCR).

In preferred embodiments, the size marker is placed in a vector between an upstream primer binding site and a downstream oligoda, allowing for amplification of the size markers of different lengths in a population of vectors containing inserts of different numbers of tandem repeats, this amplification employing a forward primer that binds the upstream primer binding site and an oligodT primer that is anchored to bind at the 5′ end of the oligoda in the vector, by means of a 3′ nucleotide that is complementary to the last nucleotide of the lower strand tandem repeat oligonucleotide.

The present invention further provides a double-stranded fragment useful as an internal control where samples of nucleic acid are to be loaded for electrophoresis, especially in a capillary electrophoreser. Inclusion of an internal control in precise amounts allows for normalization of quantitative data on amounts of different nucleic acid samples loaded into the machine, allowing for more precise relating of the measured amounts to actual amounts present. The internal control is double-stranded fragment whose upper strand is composed of the adaptor sequence upper strand, then an arbitrary sequence of any desired length, then an anchor base chosen from T, C or G, then a sequence complementary to the RT oligodT primer. The length is chosen long enough not to interfere with the fragments coming from the sample (there are many more fragments in the short range), e.g. around 470 bp.

Thus, embodiments of an internal control provided in accordance with the present invention may have the sequence:

(SEQ ID NO. 30)

5′-AGGACATTTGTGAGTCAGGCGTGTCTTGGATGC(N)ₚV(A)_{z′}ACCG
AAACAGTCCAGCGTGAATTGG-3′

wherein N is any nucleotide (A, T, C or G) and p is a number to provide a desired overall length of polynucleotide, wherein p is preferably 300-700, preferably 350-450, preferably 600-700, V′ is T, C or G, and z′ is a number 10-40, preferably 15-30, more preferably about 25. The number z′ is selected to provide an oligoA sequence complementary to the oligoT sequence in the RT primer (see SEQ ID NO. 33 and SEQ ID NO. 34). The arbitrary sequence (N)ₚ is preferably a sequence with low fragment density.

The internal control is a double-stranded molecule whose upper strand is composed of the adaptor sequence upper strand (SEQ ID NO. 31), an arbitrary sequence of any desired length, an anchor base chosen from T, C or G, and a sequence complementary to the RT primer (SEQ ID NO. 33 or SEQ ID NO. 35). The overall length is chosen to be long enough not to interfere with fragments coming from the sample, e.g. about 470 bp. The overall length in accordance with the above formula is (33+p+1+z′+25), so if z′ is 10-40 then for a fragment of overall length of about 470, p may be about 371-401. For any given number z′, complementary to the oligoT sequence in the RT primer, p can be selected accordingly for the desired overall length.

### EXPERIMENTAL EXEMPLIFICATION AND COMPARISON, AND DISCUSSION

A nested PCR system was designed, this involving testing of a large number of primer pairs, designed with the constraint that even if nested PCR was used, one of the primers in the second PCR step must be an anchored oligo-dT primer. This fixes the position of the beginning of polyadenylation sequence and gives amplified nucleic acid fragments a length defined by annealing of the adapter (and consequently primer) at the end away from the oligo-dT.

A nested PCR protocol was designed that gives superior results on complex reaction mixtures containing mRNA where only a fraction carry a ligated upstream adaptor.

Because all polymerases tested have a tendency to slip when elongating across the oligo-dT sequence, a fluorescent label when used was placed on the oligo-dT primer (placing it on the other, forward primer labels the strand which is elongated across the oligo-dT stretch and gives a stuttering split peak pattern). Nested PCR with an unlabelled first PCR overcomes the linear amplification of fragments lacking adaptor (they will be labelled in the second PCR because they have oligo-dT sequence, and they start out 256 times more abundant than the desired fragments).

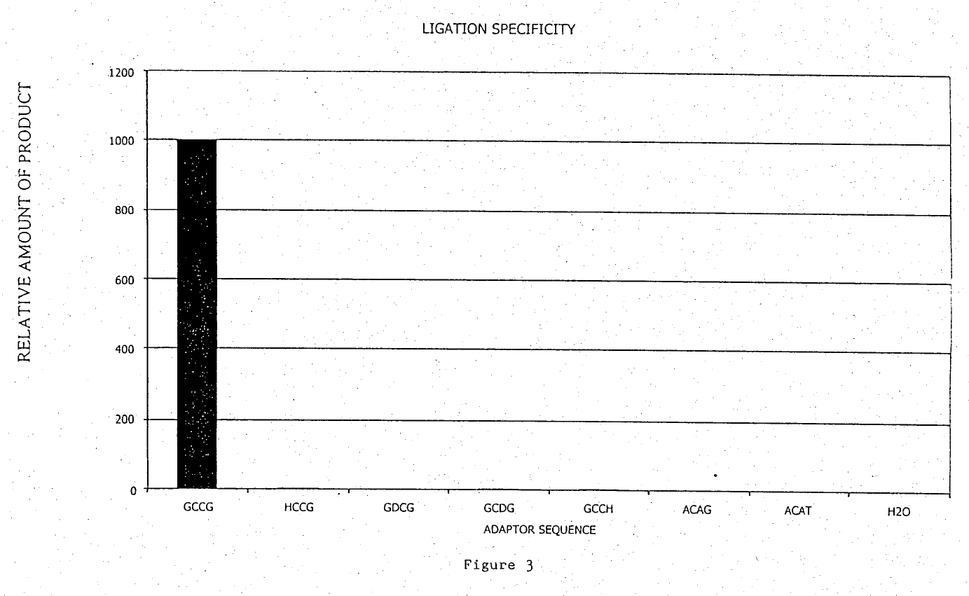

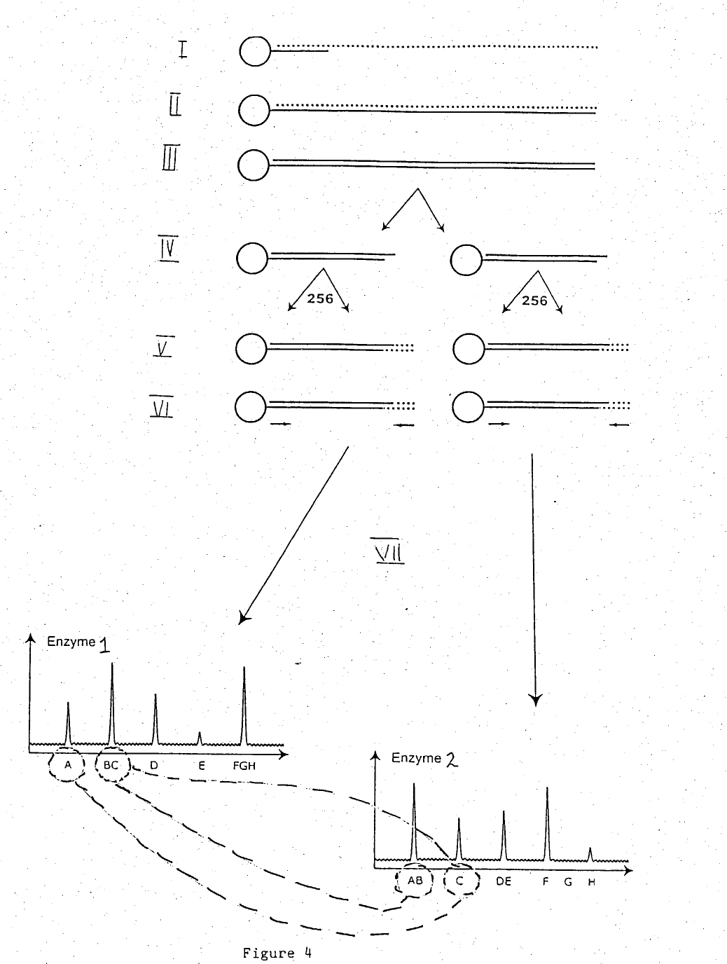

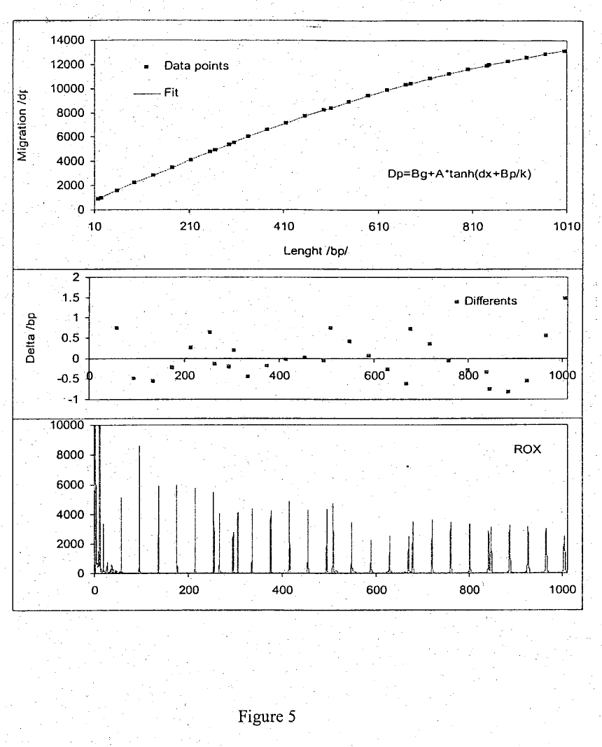

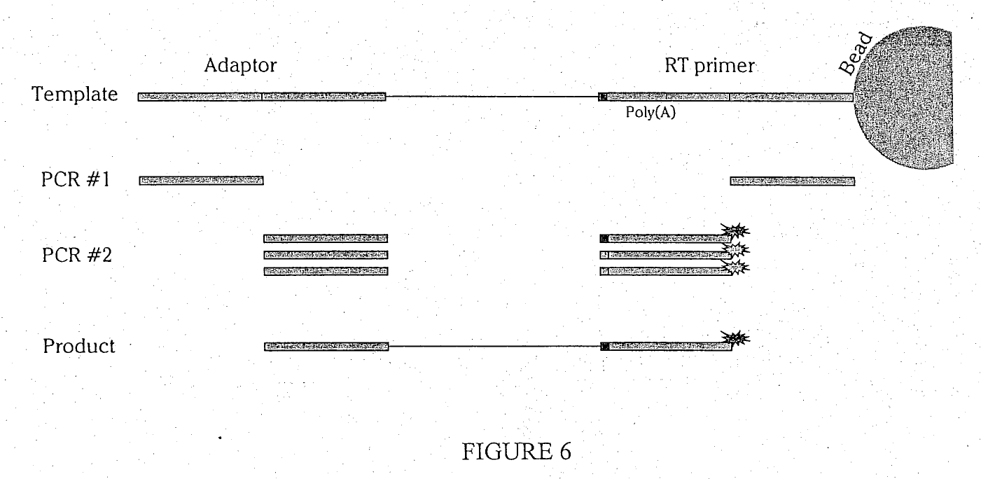

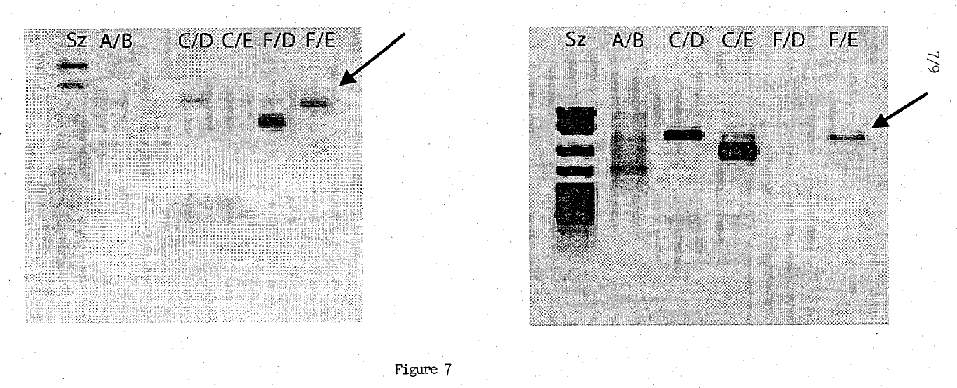

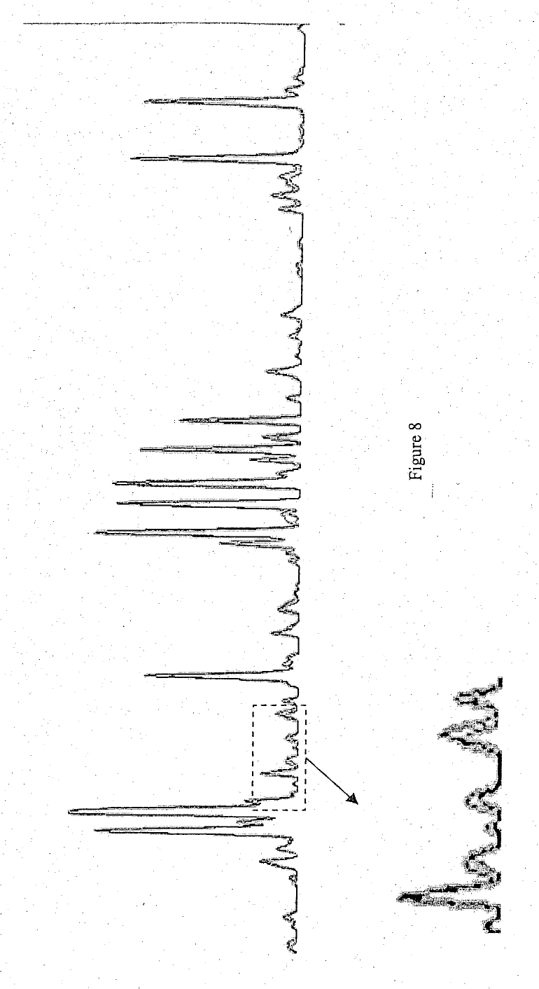

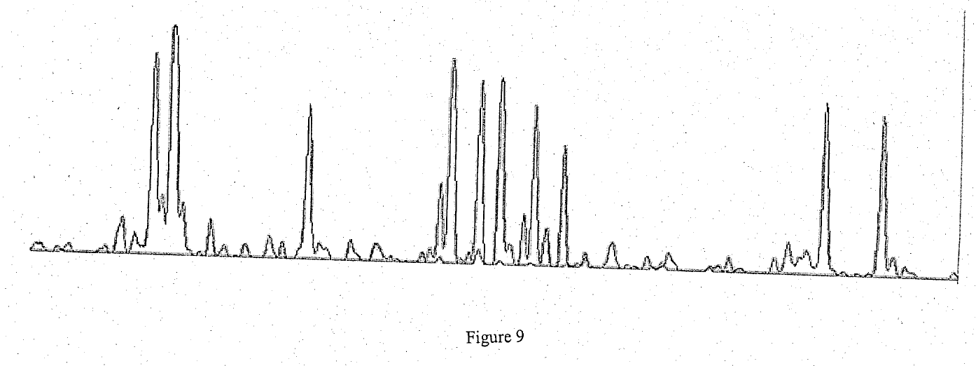

Primers for the first PCR were obtained by choosing random sequences from lambda phage DNA and the C. Tenans gene RBD). FIG. 3 shows the result of these experiments and the optimal primer pair (labelled E/F in the figure) chosen was

| | |
|---|---|
| 5′-AGGACATTTGTGAGTCAGGC-3′ and | (from lambda - SEQ ID NO. 26) |
| 5′-TTCACGCTGGACTGTTTCGG-3′. | (from RBD - SEQ ID NO. 27) |

The forward primer for the second PCR was obtained in a similar fashion by systematically varying the length of the primer described in GB0018016.6 and PCT/IB01/01539 and the optimal primer was 13 nucleotides long (5′-GTGTCTTGGATGC-3′—SEQ ID NO.35). This primer was used together with an anchored oligo-dT primer as described in the previous application: 5′-TTTTTTTTTTTTTTTTTTTTTTTTTV-3′ (SEQ ID NO. 36), i.e. (T)₂₅V, wherein V is A, C or G. 3′ anchoring in this system worked, as shown by performing Sanger sequencing-reactions on fragments carrying poly(A) tails with matched and mismatched anchors (see Table 1). As shown in the table, only anchored primers that matched the anchor of the template produce readable sequence.

Adaptors for use with Type IIS enzymes in RNA profiling in accordance with GB0018016.6 and PCT/IB01/01539 were designed to correspond to the nested PCR of the present invention:

upper strand:

(SEQ ID NO. 31)

5′-AGGACATTTGTGAGTCAGGCGTGTCTTGGATGC-3′, and
lower strand:

(SEQ ID NO. 32)

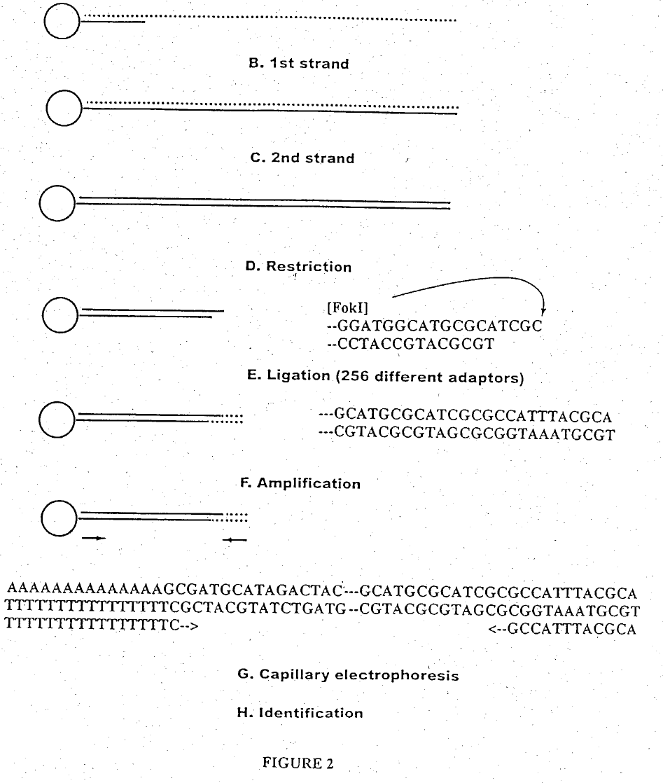

5′-pNNNNGCATCCAAGACACGCCTGACTCACAAATGTCCT-3′,

where NNNN corresponds to the 256 different possible cohesive ends (combinations of A, T, C and. G in each position) and p denotes a 5′ phosphate). The upper strand may be blocked, e.g. with a 3′ dideoxycytosine, to force ligation on the lower strand, and the lower strand may be left unphosphorylated to force ligation on the upper strand. A redesigned oligo-dT primer carrying the template sequence for the first PCR was used for reverse transcription of RNA to cDNA to enable nested PCR: 5′-CCAATTCACGCTGGACTGTTTCGG(T)_{z}-3′ (SEQ ID NO. 33), wherein z is 10-40, preferably 15-30, more preferably about 25 (this latter providing a sequence of (5′-CCAATTCACGCTGGACTGTTTCGG TTTTTTTTTTTTTTTTTTTTTTTTT-3′ (SEQ ID NO. 34), this RT primer being optionally 5′-biotinylated for use with a solid phase. A complete nested PCR system in accordance with an embodiment of the present invention is summarized in FIG. 2.

The inventors further developed a size and quantification standard designed to mimic 3′-end RNA fragments. Such fragments are often repetitive in nature and contain a polyadenylate stretch at the end. The size standard was designed by tandem ligation of arbitrary 40-mers:

(SEQ ID NO. 28)

5′-CTAGTCCTGCAGGTTTAAACGAATTCGCCCTTGGATGCCT-3′

(SEQ ID NO. 29)

3′-AGGACGTCCAAATTTGCTTAAGCGGGAACCTACGGAGATC-5′

into a vector so that the tandemly repeated sequence is inserted in the vector between an upstream primer binding site and a downstream oligo-da sequence (e.g. oligo-dA(25)) and then selecting clones with different number of inserted 40-mers. These two strands anneal to leave an overhang (CTAG) at each end. A tandomly repeated structure may be produced using ligase. From a set of such vectors, one can amplify desired fragments using an anchored oligo-dT primer (e.g. (T)₂₅C) and an upstream primer in the vector sequence. By varying the position of the upstream primer, each vector (carrying a fixed number of repeats) can generate fragments of different sizes. For example, in one embodiment a population of vectors with between 0 and 25 repeats is provided, allowing for generation in a single amplification reaction fragments spanning from 0 to 1000 bp. Several advantageous aspects of the size standard can be capitalized on:

1. Its general composition mimics that of cDNA 3′ fragments, allowing migration through capillary electrophoresis in a similar manner.

2.By co-amplifying all or some of the size standard fragments it is possible to generate a standard curve for the size- dependence of amplification efficiency. Such a curve can be used to control for this effect in each reaction for a given enzyme.

3.By co-injecting size standard fragments of known abundance with unknown fragments labelled with a different fluorescent dye, one can use the. area of each size standard peak to control for differential injection efficiencies at different fragment lengths.

The size standard was. validated by fitting a hyperbolic function to the standard curve and then computing the residuals (i.e. the local sizing error). The size standard showed sub-basepair accuracy across the entire range.

The inventors further designed an internal control for amplifying with all three anchored oligo-dT primers (i.e. if the anchoring base is A, G or C) by ligating the adaptor sequence to fragments of known length with the three different terminating nucleotides and inserting the result into a vector. This internal control can be added to the reaction prior to adaptor ligation (because it is pre-ligated) and will control for differential pipetting during all subsequent steps and capillary-to-capillary differences in loading.

FIGS. 5 and 6 summarize the quality of results obtained using this system of RNA profiling.

Use of PCR primers with one or more bases protruding into unknown sequence to generate subsets (frames)

RNA was purified according to standard techniques. The RNA was denatured at 65° C. for 10 minutes and added to Oligotex beads (Qiagen) and annealed to the oligo dT template covalently bound to the beads. A first strand cDNA synthesis was carried out using the mRNA attached to the Oligotex beads as template. This first strand cDNA therefore becomes covalently attached to the oligotex beads (Hara et al. (1991) Nucleic Acids Res. 19, 7097). Second strand synthesis was performed as described in Hara et al above. Briefly, the first strand was synthesized by reverse transcriptase (RT) from mRNA primed with oligo-dT. The second strand was produced by an RNase, which cleaves the mRNA, and a DNA Polymerase, which primes off small RNA fragments which are left by the RNase, displacing other RNA fragments as it goes along. The double-stranded cDNA attached to the Oligotex beads was purified and restriction digested with HaeII. HaeII was used. Alternative enzymes include ApoI, XjoII and Hsp921 (Type II) and FokI, BbvI and Alw261 (Type IIS). The cDNA was again purified retaining the fraction of cDNA attached to the Oligotex.

An adaptor was ligated to the HaeII site of the CDNA. The adaptor contained sequences complementary to the HaeII site and extra nucleotides to provide a universal template for PCR of all cDNAs. The cDNA was then again purified to remove salt, protein and unligated adaptors.

The cDNA was divided into 96 equal pools in a 96 well dish. In order to PCR amplify only a subset of the purified fragments in each well, a multiplex PCR was designed as follows.

The 5′ primers were complementary to the universal template but extended two bases into the unknown sequence. The first of these bases was either thymine or cytosine, corresponding to a wobbling base in the HaeII site, while the second was any of guanine, cytosine, thymine or adenosine. Each 5′ primer was fluorescently coupled by a carbon spacer to fluorochromes detectable by the ABI Prism capillary sequencer. The fluorochrome was matched to the second base. Each well received four primers with all four fluorochromes (and hence all four second bases); half of the wells received primers with a thymine first base, half with a cytosine first base.

The 3′ primers were oligo dT and therefore complementary to the polyadenylation sequence of the original mRNA. Each primer was designed with three bases extending into unknown sequence, the first of which was either guanine, adenosine or cytosine, while the other two was any of the four bases. Each well received a single 3′ primer. Thus, the PCR reaction was multiplexed into 384 sub-reactions: 96 wells with four fluorochrome channels in each.

A standard PCR reaction mix was added, including buffer, nucleotides, polymerase. The PCR was run on a Peltier thermal cycler (PTC-200). Each primer pair used in this experiment recognises and amplifies only genes containing the unique 4 nucleotide combination of that primer pair. The size of the PCR fragment of each of these genes corresponds to the length between the polyadenylation and the closest HaeII site.

The resulting PCR products were isopropanol precipitated and loaded onto an ABI prism capillary sequencer. The PCR fragments representing the expressed genes were thus, separated according to size and the fluorescence of each fragment quantitated using the detector and software supplied with the ABI Prism.

The combination of primers used lead to a theoretical mean of ˜70 PCR products in each fluorescent channel and sample (based on 20% genes expressed in a given sample and a total of 140,000 genes). Analysis of statistical size distribution of 3′ fragments including the polyadenylation generated from known genes following HaeII restriction digestion, showed that an estimated 80% can be uniquely identified based on frame and length of fragment alone. The ABI prism has 0.5% resolution between 1-2,000 nucleotides. Allowing for this uncertainty, ˜60% of the expressed genes can be uniquely identified. Using an additional parallel experiment using the same protocol but replacing the HaeII enzyme with another 5 base cutting restriction enzyme increases the theoretical limit to ˜96% and the practical limit (given the resolution of the ABI Prism) to ˜85% of all transcripts in the genome.

The level of each mRNA in the sample corresponds to the signal strength in the ABI prism. Combining the information unique to each fragment in this analysis, i.e. 8.5 nucleotides (including the HaeII recognition sequence) and the size from poly adenylation to the HaeII restriction site, the identity (EST, gene or mRNA identity) of each mRNA can thus be established. A searchable database on all known genes and unigene EST clusters was constructed as follows.

Unigene, a public database containing clusters of partially homologous fragments was downloaded (although the algorithm will work with any set of single or clustered fragments). For each cluster, all fragments containing a polyA signal and a polyA sequence were scanned for an upstream HaeII site. If no HaeII site was found, then the fragments were extended towards 5′ using sequences from the same cluster until a HaeII site was found. Then, the frame was determined from the base pairs adjacent to the HaeII and the polyA sequences and the length of a HaeII digest was calculated. The frame and length were used as indexes in the database for quick retrieval.

The output from the ABI Prism was run against the database, thus allowing the identification of expression level of all known genes and ESTs expressed in the RNA of this study. The identification in a cell or tissue of virtually all genes expressed as well as quantification of their expression levels was accomplished by a simple double-strand cDNA reaction and a 3 hour run on a 96 capillary sequencer.

Ligation of multiple adapters to cohesive ends generated by a Type IIS enzyme to generate subsets (frames), followed by PCR with universal primers

In another set of experiments the method was simplified and an increased resolution was achieved. cDNA was synthezised on solid support as described in Example 1, but this time using magnetic DynaBeads(as described in materials and methods). The cDNA was then cleaved with a class-IIS endonuclease with a recognition sequence of 4 or 5 nucleotides.

Class IIS restriction endonucleases cleave double-stranded DNA at precise distances from their recognition sequences (at 9 and 13 nucleotides from the recognition sequence in the example of the class IIS restriction endonuclease FokI). Other examples of class IIS restriction endonucleases include BbvI, SfaNI and Alw26I and others described in Szybalski et al. (1991) Gene, 100, 13-26. The 3′ parts of the cDNA were then purified using the solid support as described above. The cDNA was then divided into 256 fractions and a different adaptor was ligated to the fragments in each fraction.

For example, FokI cleavage leads to four nucleotides 5′ overhang, with each overhang consisting of a gene-specific but arbitrary combination of bases. One adaptor carrying a single possible nucleotide combination in these four positions was used in each fraction i.e. a total of 256 adapters and fractions.

Highly specific ligation of adaptors bearing a given nucleotide combination to the complementary nucleotide sequence in the fragment population was achieved by chemically blocking the adaptors on one strand, by using a deoxy oligonucleotide. As a result, ligation was forced to occur only on the other strand.

The specificity of ligation was tested using a single template, bearing a four base pair overhang. Adaptors were designed which were either exactly complementary to this overhang, or which had 1, 2 or 3 mismatches. Adaptors were ligated to the template, PCR was performed, and the relative amount of product obtained from each of the adaptor sequences was assessed.

It was found that high specificity was achieved for an adaptor blocked by including a deoxy nucleotide at the 3′ end of the upper strand (and also at the 3′ end of the lower strand in order to prevent interference at the PCR step). The results are shown in FIG. 3. The sequence GCCG is exactly complementary to the sequence of the template oligonucleotide. It can be seen that the amount of product bearing this sequence is approximately 250 times greater than the amount of product bearing sequences with one or more mismatches. Hence it can be seen that the ligation reaction proceeds with high specificity.

Adaptors which were chemically blocked by introducing at the 5′ end of the lower strand an oligonucleotide in which the phosphate group is replaced by a nitrogen group were also found to improve ligation specificity, although the degree of improvement was found to be less than with the adaptors described above.

In addition, ligation conditions which conferred high reaction efficiency were used (as described in materials and methods).

Again taking advantage of the solid support, the CDNA was then purified to remove excess non-ligated adaptor. PCR was performed on the 256 fractions using one universal primer complementary to the constant part of the adapter sequence and one complementary to the poly-A tail.

The 3′ primers were oligo dT and therefore complementary to the polyadenylation sequence of the original mRNA. Each primer was designed with a base extending into unknown sequence, guanine, adenosine or cytosine. (A second or still further base may be included, being any of guanine, adenosine, thymine or cytosine.) Each well received a mixture of the three possible 3′ primers. This ensured that the 3′ primer would always direct the polymerase to the beginning of the poly-A tail, giving a defined and reproducible fragment length.

The advantage of this second protocol is that the splitting into multiple frames occurs at the ligation step, not the PCR, allowing the use of high-stringency universal primers in the PCR. This leads to improved specificity and reproducibility. Another advantage is that a set of 256 adapters compatible with any 4-base overhang can be reused in multiple experiments with Type IIS enzymes which recognize different sequences but still give four base overhangs. Thus for each length of overhang, a single set of adapters will suffice.

The resulting PCR products were purified and loaded onto an ABI prism capillary sequencer. The PCR fragments representing the expressed genes were thus separated according to size and the fluorescence of each fragment quantified using the detector and software supplied with the ABI Prism.

Four separate frames may be run in each reaction vessel using different fluorophores because the ABI Prism has four detection channels. Four different universal forward primers (5′ end) have been designed with no cross-hybridization between them. The use of these primers allowed the 256 reactions to be reduced to 64. In an alternative embodiment, three primers and three adaptors are employed, allowing for one channel in the ABI Prism to be used for a size reference. The total number of reactions is then 86.

It is also desirable to increase the annealing temperature of the oligo-dT primer. This was enabled by adding a tail with an arbitrary sequence (not cross-hybridizing with any of the forward primers) and mixing the long primer containing oligo-dT with a short primer identical with the arbitrary sequence and having a high melting point. The first few cycles were then be performed at low temperature, at which only the oligo-dT primers anneal, after which all fragments had the tail added. This then allowed for subsequent cycles to be performed at higher temperature (at which only the short primer anneals) relying on the longer tail being present. This approach increases specificity of PCR and reduces background.

The combination of primers used leads to a theoretical mean of ˜80 PCR products in each fluorescent channel and sample (based on 20% genes expressed in a given sample and a total of 100 000 transcripts). Analysis of statistical size distribution of 3′ fragments including the polyadenylation generated from known genes following FokI restriction digestion, provides that an estimated 67% can be uniquely identified based on frame and length of fragment alone. Using an additional parallel experiment using the same protocol but replacing the FokI enzyme with another 5 base cutting class IIS restriction enzyme increases the theoretical limit to ˜89%; a third experiment yields ˜99% of all transcripts in the genome.

These numbers are under-estimates since in practice a gene that runs as a doublet in two experiments can still be identified as unique if at least one of its doublet partners is not expressed (a 96% chance) using the combinatorial algorithms of this invention. This and similar effects have been disregarded in the above calculations.

Combining the information unique to each fragment in this analysis, i.e. 9 nucleotides (including the FokI recognition sequence and cleavage site) and the size from polyadenylation to the FokI restriction site obtained from the capillary sequencer, the identity (EST, gene or mRNA identity) of each mRNA can thus be established. A searchable database on all known genes and unigene EST clusters was constructed as described above.

Fragment identification

Combinatorial algorithms of the invention, based on multiple independent patterns for a sample, offer a number of advantages for gene identification.

Firstly, the more experiments are performed the likelier it is that a given gene runs as a singlet fragment in at least one of them and can thus be unambiguously identified. Even if a given gene runs as a doublet in all experiments, it can still be identified if one of its doublet partners in one of the experiments should run as a singlet in another experiment and is absent there.

For example, if there is a fragment in experiment I at 162 bp corresponding to genes A and B, and one in experiment II at 367 bp corresponding to A and C, then one can look up C in experiment I (if it should run as a singlet there, say at 214 bp, and it is absent, i.e. there is no peak at 214 bp, then the peak at 162 bp in I can be identified as A) and B in experiment II. This simple procedure greatly increases the number of genes which can be unambiguously identified even when only two experiments have been performed.

Computer simulations using estimated error rates from an ABI Prism capillary electrophoresis machine indicate that 85-99% of all genes can be correctly identified even in the presence of normal fragment length errors.

Secondly, both of these combinatorial algorithms can be used to overcome uncertainties about fragment sizes or gene 3′-end lengths. This is because as long as the number of fragment peaks obtained from the sample plus the number of genes which can be eliminated as definitely not expressed is greater than the total number of candidate genes (i.e., the number of genes in the organism), the algorithms will be successful in assigning a gene to each fragment. In terms of the mathematical form of the algorithm, the system can be solved if the number of equations is greater than the number of candidate genes.

Thus, the number of candidate genes.can be increased, up to a point, without losing the ability to successfully choose the correct candidate for each fragment. In cases where the length of the fragment is unknown, matches to fragments having each of the possible fragment lengths can be added to the list of genes which may be present. Similarly, when the position of the 3′ end in the database is unknown, all genes which could have a 3′ end in the position indicated by the fragment can be added to the list of genes which may be present. The false positives are subsequently eliminated automatically by the algorithm, provided the above condition is fulfilled.

The power of the system to eliminate false positives can be increased by performing greater numbers of independent profiles, as this will increase both the number of fragments and the number of genes which can be eliminated as definitely not present.

The optimum number of subdivisions can be determined.

The purpose of subdividing the reaction is to reduce the number of fragment peaks which correspond to multiple genes.

Two factors determine the number of doublets: the number of sub-reactions and the size distribution of fragments.

The optimal size distribution depends on the detection method. Capillary electrophoresis has single-basepair resolution up to 500 bp and about 0.15% resolution after that. Thus a distribution extending too far would not be useful. But a narrow distribution may present difficulties as well, because then genes will begin to run as true doublets (with the exact same length) which cannot be resolved no matter what the resolution.

The probability of finding a fragment of length n if you cut with an enzyme which cuts with a probability 1/512 is

P₁(n)=(511/512)ⁿ(1/512)

If the reaction is divided in 192 sub-reactions, the probability of finding a fragment of length n in a given subreaction is

P₂(n)=(511/512)ⁿ(1/512)(1/192)

The probability of this fragment corresponding to a single gene from M possible genes is

P_{unique}(n)=P₂(n) (1-P₂ (n))^{(M-1)}

In other words, this is the probability that one gene gives a fragment of that length and all others do not.

The total number of genes which can be uniquely identified in a single experiment can be obtained by summing over all detectable lengths.

Taking instrument imprecision into account, P_{unique} becomes

P_{unique}(n)=P₂(n) ((1-P₂(n))^{(M-1)})^{(1+2En)}

where E is the magnitude of the imprecision. This states that a unique gene can be identified if no other gene has the same length +/−a factor E.

For example, if there are 50 000 genes in the human, our instrument has an error of 0.2% and can detect fragments up to 1000 bp, and we cut with an enzyme which cuts 1/512 of all sequences, subdividing in 192 subreactions, then we can identify 56% of all genes uniquely in a single experiment, 80% in two and 96% in three.

In Mathematica, the number of uniquely identifiable genes can be calcuated as follows:

Prob[n_]:=(511/512)n*1/512*1/192

Sum[50000*Prob[n]((1-Prob[n])50000)1+0.002n),

{n, 1, 1000}]*192

By varying the parameters one can quickly see the effects on identification probabilities.

As noted above, if more experiments are performed, more powerful combinatorial identification methods can be used, but they all benefit from an increased number of singleton genes.

### MATERIALS AND METHODS

In the following, the original primers are described as also in GB0018016.6 and PCT/IB01/01539. Thus, primers A and B are used for PCR, priming from the adaptors. In accordance with embodiments of the present invention, primer pair E and F may be used instead, especially in combination with the adaptors and/or other primers disclosed herein as components of aspects of the present invention.

Section 1—employing Type II restriction enzyme

Isolating mRNA from total RNA

Isolate mRNA from 20 ug total RNA according to Oligotex protocol until pure mRNA is bound to the beads and washed clean. Spin down and resuspend in 20 ul distilled water. The suspension should contain 0.5 mg Oligotex.

Split the reaction in 2×10 ul. Heat denature at 70° C. for 10 min, then chill quickly on ice. Synthesize first strand cDNA using each of the protocols below:

First strand cDNA synthesis using AMV

Add first-strand buffer: 5 ul 5×AMV buffer, 2.5 ul 10 mM dNTP, 2.5 ul 40 mM NaPyrophosphate, 0.5 ul RNase inhibitor, 2 ul.AMV RT, 2.5 ul 5 mg/ml BSA.

Incubate at 42° C. for 60 min. Total volume: 25 ul. [Note: it may be better to run in 100 ul, to get a more dilute Oligotex suspension]

Second strand cDNA synthesis using AMV

Add 12.5 ul lOx AMV second-strand buffer (500 mM Tris pH 7.2, 900 mM KCl, 30 mM MgCl2, 30 mM DTT, 5 mg/ml BSA), 29 U E Coli DNA Polymerase I, 1 U RNase H to a final volume of 125 ul with dH2O.

Incubate at 14° C. for 2 hours.

Restriction enzyme cleavage and dephosphorylation

Spin down Oligotex/cDNA complexes and resuspend in 1.8 ul 10×FokI buffer, 16.2 ul H2O, 2 ul FokI, 1 u Calf Intestinal Phosphatase (included to dephosphorylate cohesive ends to prevent self-ligation in the next step).

Incubate at 37° C. for 1 hour.

Spin down and remove supernatant for quality-control.

Phosphatase deactivation

Add 70 ul TE. Heat to 70° C. for 10 minutes. Cool down to room temperature and leave for 10 minutes.

Ligation

Resuspend in 2 ul 10× ligation buffer, 100× adaptor, 2 ul ligase, H₂O to 20 ul.

Incubate at RT for 2 hours.

Spin down and wash with 10 mM Tris (pH 7.6).

Primer and adaptor design

The adaptor is as follows (shown 5′ to 3′). It consists of a long and-a short strand which are complementary. The long strand has four extra bases complementary to the GCGC cohesive end generated by the HaeII enzyme cleavage.

| | |
|---|---|
| 5′-GTCCTCGATGTGCGC-3′ | (SEQ ID NO. 1) |
| 5′-ACATCGAGGAC-3′ | (SEQ ID NO. 2) |

The 5′ primers are 5′-GTCCTCGATGTGCGCWN-3′ (SEQ ID NO. 3), where W is A or T and N is A, C, G or T. There are 8 different 5′ primers, labelled with a fluorochrome corresponding to the last base.

The 3′ primers are T₂₅VNN, where V is A, G or C and N is A, G, C or T. That is, 25 thymines followed by three bases as shown. There are 48 different 3′ primers.

All combinations of 3′ and 5′ primers are used, or 384 in total. The 5′ primers are pooled with respect to the last base (i.e. all four fluorochromes are run in the same reaction), giving a total of 96 reactions.

The primer combinations are predispensed into 96-well PCR plates.

PCR amplification

Resuspend in 768 ul PCR.buffer (buffer, enzyme, DNTP), add 8 ul to each well of a premade primer-plate containing 2 ul primer-mix (four 5′ primers and one 3′ primer) per well.

Using hot-start touchdown PCR, amplify each fraction as follows:

Hot start

Heat to 70° C.

Add Taq polymerase

10 cycles

94° C. 30 s

60° C. 30 s, reduced by 0.5° C. each cycle

72° C. 1 min

25 cycles

94° C. 30 s

55° C. 30 s

72° C. 1 min

Finally

72° C. 5 min

Cool down to 4° C.

The touchdown ramp annealing temperature may have to be adjusted up or down. The reaction should only proceed until the plateau phase has been reached; the 25 cycles may have to be adjusted.

A rotating real-time PCR apparatus is preferred, to minimize temperature variation and to allow monitoring the plateau phase. With such a machine, Taq polymerase is loaded in the cap of each tube and the hot start is performed before the rotor is started, melting away the second strand from the Oligotex. When the-rotor starts, the beads and the first strand are pelleted and Taq drops into the reaction mix at the same time.

Quantification by capillary electrophoresis

Load the 96-well plate on an ABI Prism 3700 setup for fragment analysis with a long capillary and long run time. The output is a table of fragment length (in base pairs) and peak height/area for each peak detected.

Proceed to identification, e.g. as described above with reference to a database.

Section 2—employing Type IIS restriction enzyme

Preparation of streptavidin Dynabeads (attaching the oligos to the beads)

Wash 200 μl Dynabeads twice in 200 μl B&W buffer (Dynabeads) and then resuspend the beads in 400 μl B&W buffer.

Suspend 1250 pmol biotine T25 primer in 400 μl H₂O and mix with the beads. Incubate at RT for 15 min. Spin briefly, then remove 600 μl of the supernatent. Dispense the beads and place on a magnet for at least 30 seconds.

Wash beads twice with 200 μl B&W, and then resuspend in ²⁰⁰p¹ B&W buffer.

Binding the mRNA to the beads from total RNA Transfer 200 μl of resuspended beads into a 1.5 ml Eppendorf tube. Place on a magnet at least for 30 sec. Remove the supernatant and r.esuspend in 100pl of binding buffer(20 mM Tris-HCl, pH 7,5; 1,0 M LiCl; 2 mM EDTA). Repeat washing, and resuspend the beads in 100 μl of binding buffer.

Adjust ˜75 μg of total RNA or 2.5 μg of mRNA to 100 μl with Rnase free water or 10 mM Tris-HCl. Heat to 65° C. for 2 min.

Mix the beads thoroughly with the preheated RNA solution. Anneal by rotating or otherwise mixing for 3-5 min at room temperature (rt). Place on-a magnet for at least 30 sec. Wash twice with 200 μl of washing buffer B (lOmM Tris-HCL pH7.5;.0.15 MliCl; 1 mM EDTA).

First strand synthesis

Wash the beads at least twice with 200 μl 1× AMV buffer (Promega) using the magnet as described previously. Mix together 5 μl 5× AMV buffer; 2.5 μl 10 mM DNTP; 2.5 μl 40 mM Na pyrophosphate; 0.5 μl RNase inhibitor; 2 μl AMV RT (Promega); 1.25 μl 10 mg/ml BSA; 11.25 μl H₂O (Rnase free) (Total volume 25 μl). Resuspend the beads in this mixture.

Incubate at 42° C. for 1 h, with mixing.

Second strand synthesis

Add 100 μl of second strand mixture (6.25 μl 1M Tris pH 7.5; 11.25 μl 1M KCl; 15 μl MgCl₂; 3.75 μl DTT; 6.25 μl BSA; 1 μl Rnase H, 3 μl DNA pol I; 53.5 μl H₂O) (total volume 100 μl) directly to the 1^{st} strand reaction.

Incubate at 14° C. for 2 h, with mixing.

Cleavage

Wash the beads on magnet 2× with TE (10 mM TRIS, 1 mM EDTA, pH 7.5) and 2× with 100-200 μl NEB buffer. Resuspend in 30 pl of NEB buffer

Add 1 μl of the appropriate Type IIS enzyme and mix.

Incubate at 37° C. for 1-2 h, mixing frequently. Wash three times with TE in 1350 μl using the magnet as described above, and then twice with 1350 l 2× ligation buffer.

Resuspend in 1606 μl 2× ligase buffer with ligase enzyme.

Adapter ligation (in 256 different vessels)

Aliquot 6 μl of cut template per well in 256 wells containing 30 pmol adaptor in 4 μl for a total volume of 10 μl. Incubate 1 h at 37° C. with mixing. Wash in TE 80 μl 2× and dilute in 20 μl H₂O

Adaptor and primer design

The adaptors in these embodiments are as follows (shown 5′ to 3′). Each pair is composed of a short and a long strand, which are complementary. The long strands have four nucleotides complementary to the cohesive ends generated by the FokI cleavage (a total of 4×4×4×4=256 possible adapters).

Labelled versions of the upper, shorter strands also serve as forward PCR primers.

| | |
|---|---|
| 5′-CCAAACCCGCTTATTCTCCGCAGTA-3′ | (SEQ ID NO. 4) |
| 5′-NNNNTACTGCGGAGAATAAGCGGGTTTGG-3′ | (SEQ ID NO. 5) |
| 5′-GTGCTCTGGTGCTACGCATTTACCG-3′ | (SEQ ID NO. 6) |
| 5′-NNNNCGGTAAATGCGTAGCACCAGAGCAC-3′ | (SEQ ID NO. 7) |
| 5′-CCGTGGCAATTAGTCGTCTAACGCT-3′ | (SEQ ID NO. 8) |
| 5′-NNNNAGCGTTAGACGACTAATTGCCACGG-3′ | (SEQ ID NO. 9) |

Each of the adaptors is be blocked on one strand. This may be achieved by blocking the upper strand-at the 3′ end using a deoxy (dd) oligonucleotide, as shown below.

(SEQ ID NO. 4)

5′ (OH)-CCAAACCCGCTTATTCTCCGCAGTddA-3′

(SEQ ID NO. 5)

5′ (P)-NNNNTACTGCGGAGAATAAGCGGGTTTGG-(OH)-3′

(SEQ ID NO. 6)

5′ (OH)-GTGCTCTGGTGCTACGCATTTACCddG-3′

(SEQ ID NO. 7)

5′ (P)-NNNNCGGTAAATGCGTAGCACCAGAGCAC-(OH)-3′

(SEQ ID NO. 8)

5′ (OH)-CCGTGGCAATTAGTCGTCTAACGCddT-3′

(SEQ ID NO. 9)

5′ (P)-NNNNAGCGTTAGACGACTAATTGCCACGG-(OH)-3′

Alternatively, blocking may be achieved by replacing the phosphate group at the 5′ end of the lower strand with a nitrogen, hydroxyl, or other blocking moiety.

The reverse primers are as follows

(SEQ ID NO. 10)

5′-CTGGGTAGGTCCGATTTAGGCTTTTTTTTTTTTTTTTTTTTTV-3′

(SEQ ID NO. 11)

5′-CTGGGTAGGTCCGATTTAGGC-3′

where V=A, C or G, for a total of three long reverse primers.

Universal PCR

Add 18 ul PCR buffer (buffer, enzyme, dNTP, three universal adapter primers, anchored oligo-T primers).

Amplify each fraction as follows:

Hot start

Heat

Add Taq at 70° C.

(or use heat-activated Taq)

2 cycles

94° C. 30 s50° C. 30 s 72° C. 1 min

25 cycles

94° C. 30 s61° C. 30 s72° C. 1 min

Finally

72° C. 5 min

Cool down to 4° C.

A rotating real-time PCR apparatus is preferred, to minimize temperature variation and to allow monitoring the plateau phase. With such a machine, Taq polymerase is loaded in the cap of each tube and the hot start is performed before the rotor is started, melting away the second strand from the Oligotex. When the rotor starts, the beads and the first strand are pelleted and Taq drops into.the reaction mix at the same time.

Quantification by capillary electrophoresis

Load the 96-well plate on an ABI Prism 3700 setup for fragment analysis with a long capillary and long run time. The output will be a table of fragment length (in base pairs) and peak height/area for each peak detected.

### DISCUSSION

Most microarrays (except Affymetrix) are based on hybridisation. to spotted cDNAs on a glass or membrane surface. This requires cloning, amplification and spotting of the cDNA of each gene in the genome for a comparable analysis to what can be performed in under one day using embodiments.of the present invention.

All microarrays require the prior knowledge of each gene such as the cloning and sequencing of cDNAs or an expressed sequence tag. Embodiments of the present invention allow identification and quantification of all genes expressed in the genome without any prior information on their existence.

The Affymetrix microarray which at present allows quantification of expression of the largest number of genes in mammals cover at most 32,000 genes. Embodiments of the present invention can be applied to all genes in the genome.

All microarray-based technologies are limited to the species the array is generated from and depend on an availability of sequence information for the species of interest. Embodiments of the present invention can be applied to all species from plants to mammals without any prior cDNA or DNA sequence information.

Microarrays are often unable to differentiate between splice variants, and are always unable to detect rare alleles. Embodiments of the present invention allow for detection of the actual transcripts present in the sample.

All microarray-based technologies are based on indirect measurement of quantities following DNA hybridisation. Real copy numbers can be quantitated using the present invention.

Hybridization-based technologies depend on the highly unpredictable and non-linear nature of hybridization kinetics; embodiments of the present invention employ the exponential, reproducible competitive polymerase chain reaction.

Because embodiments of the present invention are based on a kind of competitive PCR, i.e. all fragments in a reaction are amplified by the same primer pair (or a small number of very similar primer pairs), errors are minimized. The invention allows the skilled worker to reproducibly detect about 2-fold differences in gene expression across a wide dynamic range (about 2.5 orders of magnitude); very competitive with other technologies.

Because embodiments of the present invention are PCR-based, sensitivity can be traded for starting material. In other words, it is possible to start with a smaller amount of RNA and run a few extra PCR cycles. Because PCR is exponential, an extra cycle will. cut material requirement in half while adding only about 2-3% to the experimental variation. Useful data can thus be produced from as little as a few or even single cells, while accuracy can be increased using larger samples.

Microarray-technology allowing quantification of gene expression of a significant percent of the genes is very expensive. Affymetrix microarrays covering a claimed 32,000 unique ESTs cost 4000 USD/experiment.

### REFERENCES

Alizadeh et al. (2000) Nature 403, 503-511.

Alwine et al. (1977) Proc. Natl. Acad. Sci. USA 74, 5350-5354.

Berk and Sharp (1977) Cell 12, 721-732.

Bowtell (1999) [published erratum appears in Nat Genet 1999

Feb;21(2):241]. Nat Genet 21, 25-32.

Britton-Davidian et al. (2000) Nature 403, 158.

Brown and Botstein (1999) Nat Genet 21, 33-7.

Cahill et al. (1999) Trends Cell Biol 9, M57-60.

Cho et al. (1998) Mol Cell 2, 65-73.

Collins et al. (1997) Science 278, 1580-1.

Der et al. (1998) Proc Natl Acad Sci U S A 95, 15623-8.

Duggan et al. (1999) Nat Genet 21, 10-4.

Golub et al. (1999) Science 286, 531-7.

Iyer et al. (1999) Science 283, 83-7.

Lander (1999) Nat Genet 21, 3-4.

Lengauer et al. (1998) Nature 396, 643-9.

Liang and Pardee (1992) Science 257, 967-71.

Lipshutz et al. (1999). High density synthetic oligonucleotide arrays. Nat Genet 21, 20-4.

McCormick (1999) Trends Cell Biol 9, M53-6.

Okubo et al. (1992) Nat Genet 2, 173-9.

Paabo (1999) Trends Cell Biol 9, M13-6.

Perou et al. (1999) Proc Natl Acad Sci U S A 96, 9212-7.

Schena et al. (1995) Science 270, 467-70.

Schena et al. (1996) Proc Natl Acad Sci U S A 93, 10614-9.

Southern et al. (1999) Nat Genet 21, 5-9.

Stoler et al. (1999) Proc Natl Acad Sci U S A 96, 15121-6.

Szallasi (1998) Nat Biotechnol 16, 1292-3.

Thomson and Esposito (1999) Trends Cell Biol 9, M17-20.

Velculescu et al. (1995) Science 270, 484-7.

The following are preferred embodiments of the present invention, in which any combination of one or more of the primers of the invention, the size standard of the invention and/or the internal control may be used:

1. An embodiment which is a method of providing a profile of mRNA molecules present in a sample, the method comprising:

synthesizing a cDNA strand complementary to each mRNA using the mRNA as template, thereby providing a population of first cDNA strands;.

removing the mRNA;

synthesizing a second cDNA strand complementary to each first strand, thereby providing a population of double-stranded cDNA molecules;

digesting the double-stranded cDNA molecules with a Type II or Type IIS restriction enzyme to provide a population of digested double-stranded cDNA molecules, each digested double-stranded cDNA molecule having a cohesive end provided by the restriction enzyme digestion;

ligating a population of adaptor oligonucleotides to the cohesive end of each of the digested double-stranded cDNA molecules, the adaptor oligonucleotides each comprising an end sequence complementary to a cohesive end and a primer annealing sequence, thereby providing double-stranded template cDNA molecules each comprising a first strand and a second strand wherein the first strand of the double-stranded template cDNA molecules each comprise a 3′ terminal adaptor oligonucleotide and the second strand of the double-stranded template cDNA molecules each comprise a 3′ terminal polyA sequence;

purifying said double-stranded template cDNA molecules;

performing polymerase chain reaction amplification on the double-stranded template CDNA molecules having a sequence complementary to a 3′ end of an mRNA using a population of first primers and a population of second primers,

wherein the first primers each comprise a sequence which anneals to a primer annealing sequence of an adaptor oligonucleotide; and

where the restriction enzyme is a Type II enzyme the first primers each comprise at least one 3′ terminal variable nucleotide and optionally.more than one 3′ terminal variable nucleotides wherein the variable nucleotide is, or at a corresponding position within the variable nucleotides each first primer has, a nucleotide selected from A, T, C and G, whereby-the population of first primers primes synthesis in the polymerase chain reaction of first strand product DNA molecules each of which is complementary to the first strand of a template cDNA molecule that comprises adjacent to the primer annealing sequence within the first strand of the template cDNA molecule a nucleotide or sequence of nucleotides complementary to the variable nucleotide or nucleotides of a first primer within the population of first primers; or

where the restriction enzyme is a Type IIS enzyme the first primers prime synthesis in the polymerase chain reaction of first strand product DNA molecules each of which is complementary to the first strand of a template cDNA molecule that comprises within the first strand of the template cDNA molecule a sequence of nucleotides complementary to an end sequence of an adaptor oligonucleotide in the population of adaptor oligonucleotides;

the second primers comprise an oligoT sequence and a 3′ variable portion conforming to the following formula: (G/C/A) (X)ₙ wherein X is any nucleotide, n is zero, at least one or more than one; whereby the population of second primers primes synthesis in the polymerase chain reaction of second strand product DNA molecules each of which is complementary to the second strand of a template cDNA molecule that comprises adjacent to polyA within the second strand of the template cDNA molecule a nucleotide or nucleotides complementary to the variable portion of a second primer within the population of second primers;

whereby the polymerase chain reaction amplification provides a population of double-stranded product DNA molecules each of which comprises a first strand product DNA molecule and a second strand product DNA molecule;

separating double-stranded product DNA molecules on the basis of length; and

detecting said double-stranded product DNA molecules;

whereby a pattern for the population of mRNA molecules present in the sample is provided by combination of length of said double-stranded product DNA molecules and (i) first primer variable nucleotide or nucleotides, where a Type II restriction enzyme is employed, or (ii) adaptor oligonucleotide end sequence, where a Type IIS restriction enzyme is employed.

In such an embodiment where a nested PCR is performed as disclosed, the first and second primers referred to are as used in the second PCR of the nested PCR (and may be referred to as second forward primers and second back primers, respectively) being preceded by a first PCR in which first forward primers and first back primers are used to provide templates for the second PCR. In the first PCR a first forward primer is used that anneals to a 3′ portion of the lower strand of the cohesive adaptor oligonucleotides, while a back primer is used that anneals to a 3′ portion of the upper strand of an adaptor extending from the polyA region.

2. An embodiment that further comprises:

generating an additional pattern for the sample using a second, different Type II or Type IIS restriction enzyme, and comparing the patterns generated using at least two different Type II or Type IIS restriction enzymes in separate experiments with a database of signals determined or predicted for known mRNA's.

3. An embodiment wherein patterns generated using at least two different Type II or Type IIS restriction enzymes in separate experiments with a database of signals determined or predicted for known mRNA's by:

(i) listing all mRNA's in the database which may correspond to a double-stranded product DNA in each experiment, forming a list of mRNA molecules possibly present for each experiment, and

(ii) for each experiment listing mRNA's which definitely do not correspond to a double-stranded product DNA molecule, forming a list of mRNA molecules definitely not present for each experiment, then

(iii) removing the mRNA molecules definitely not present from the list of mRNA molecules possibly present for each experiment, and

(iv) generating a list of mRNA molecules possibly present and mRNA molecules definitely not present by combining each list generated for each experiment in (iii);

thereby providing a profile of mRNA molecules present in the sample.

4. An embodiment which comprises comparing the patterns generated using at least two different Type II or Type IIS restriction enzymes in separate experiments with a database of signals determined or predicted for known mRNA's, by:

(i) listing all mRNA's in the database which may correspond to a double-stranded product DNA in each experiment, and forming a set of equations of the form Fi=m₁+m₂+m₃, wherein Fi is the intensity of the signal from the fragment, the numerals are the mRNA identity and wherein each mRNA which may correspond to a double-stranded product DNA appears as a term on the right-hand side;

(ii) for each experiment listing mRNA's which definitely do not correspond to double-stranded product DNA in each experiment, and writing for each gene which definitely does not correspond to a double-stranded product DNA in each experiment an equation of the form 0=m₄, wherein the numeral is the mRNA identity;

(iii) combining the sets of equations to form a system of simultaneous equations wherein the number of equations is greater than the number of genes in the organism;

(iv) determining an estimate of the expression level of each gene by solving the system of simultaneous equations,

thereby providing a profile of mRNA molecules present in the sample.

5. An embodiment comprising purifying digested double-stranded cDNA molecules which comprise a strand comprising a 3′ terminal polyA sequence, prior to ligating the adaptor oligonucleotides (cohesive adaptor oligonucleotides).

6. An embodiment comprising:

i)immobilising mRNA molecules in the sample on a solid support by annealing a polyA tail of each mRNA molecule to polyT oligonucleotides attached to a support, prior to synthesizing said first cDNA strand, removing the mRNA, and synthesizing said second cDNA strand, thereby providing a population of double-stranded cDNA molecules attached to the support; and

ii) following digesting the double-stranded cDNA molecules to provide a population of digested double-stranded cDNA molecules attached to the support, purifying the digested double-stranded cDNA molecules attached to the support by washing away material not attached to the support, prior to ligating said population of adaptor oligonucleotides to the cohesive end of each of the digested double-stranded cDNA molecules; and

iii) following ligating a population of adaptor oligonucleotides to the-cohesive end of each of the digested double-stranded cDNA molecules to provide said double-stranded cDNA template molecules, purifying the double-stranded template cDNA molecules by washing away material not attached to the support, prior to performing said polymerase chain reaction amplification on the double-stranded cDNA molecules.

7. An embodiment wherein the restriction enzyme cuts double-stranded DNA with a frequency of cutting of 1/256-1/4096 bp.

8. An embodiment wherein the frequency of cutting is 1/512 or 1/1024 bp.

9. An embodiment wherein the restriction enzyme is a Type II restriction enzyme.

10. An embodiment wherein the restriction enzyme digests double-stranded DNA to provide a cohesive end of 2-4 nucleotides.

11. An embodiment wherein the restriction enzyme is selected from the group consisting of HaeII, ApoI, XhoII and Hsp 921.

12. An embodiment wherein the first primers (second forward primers) each have one variable nucleotide.

13. An embodiment wherein the first primers (second forward primers) each have two variable nucleotides, each of which may be A, T, C or G.

14. An embodiment wherein the first primers (second forward primers) each have three variable nucleotides, each of which may be A, T, C or G.

15. An embodiment wherein each first primer (second forward primer) is labelled with a label to indicate which of A, T, C and G is said variable nucleotide or is present at said corresponding position within the variable nucleotides of the first primer (second forward primer).

16. An embodiment wherein the restriction enzyme is a Type IIS restriction enzyme.

17. An embodiment wherein the restriction enzyme digests double-stranded DNA to provide a cohesive end of 2-4 nucleotides.

18. An embodiment wherein the restriction enzyme is selected from the group consisting of FokI, BbvI, SfaNI and Alw261.

19. An embodiment wherein adaptor oligonucleotides in the population of adaptor oligonucleotides are ligated to cohesive ends of digested double-stranded cDNA molecules in separate reaction vessels from different adaptor oligonucleotides with different end sequences.

20. An embodiment wherein each reaction vessel contains a single adaptor-oligonucleotide end sequence.

21. An embodiment wherein each reaction vessel contains multiple adaptor oligonucleotide end sequences, each adaptor oligonucleotide sequence in a reaction vessel comprising a different end sequence and primer annealing sequence from the end sequence and primer annealing sequence of other adaptor oligonucleotide sequences in the same reaction vessel, corresponding multiple first primers being employed in the polymerase chain reaction amplification in each reaction vessel.

22. An embodiment wherein.n is 0.

23. An embodiment wherein n is 1.

24. An embodiment wherein n is 2.

25. An embodiment wherein first primers (second forward primers) or second primers (second back primers) are labelled.

26. An embodiment wherein the labels are fluorescent dyes readable by a sequencing machine.

27. An embodiment wherein double-stranded DNA molecules are separated on the basis of length by electrophoresis on a sequencing gel or capillary, and the pattern is generated as an electropherogram.

28. An embodiment wherein a first profile of the mRNA molecules present in a first sample is compared with a second profile of the mRNA molecules present in a second sample.

29. An embodiment wherein a difference is identified between said first profile and said second profile.

30. An embodiment wherein a nucleic acid whose expression leads to the difference between said first profile and said second profile is identified and/or obtained.

31. An embodiment wherein the presence in the sample of a known mRNA is identified.

TABLE 1

Determining anchoring specificity. Six different clones (rows) carrying a polyadenylation tail with the indicated anchor base (first column) were sequenced using anchored primers (indicated in top row). +indicates good sequences, −indicates absence of sequence. In no case did an anchored primer produce a product from a clone with a mismatched anchor. T3 and T7 primers were used as positive controls.

TABLE 1
PCR #2
Anchoring Specificity
Regular sequencing performed with anchored primers
+ good sequence
− no detectable sequence
Anchor Primer

| | | A | G | C | T3 | T7 |
|---|---|---|---|---|---|---|
| Clone | A | + | − | − | + | + |
| Poly(A) Site | A | + | − | − | + | + |
| | A | + | − | − | + | + |
| | G | − | + | − | + | + |
| | C | − | − | + | + | + |
| | C | − | − | + | + | + |

1. A method of providing a population of double-stranded product DNA molecules, the method comprising:
annealing polyA tails of mRNA molecules in a sample to an oligoT adaptor, which oligoT adaptor comprises a 3′ oligoT portion and a 5′ first back primer annealing sequence, synthesizing a cDNA. strand complementary to the mRNA molecules using the mRNA molecules as template, thereby providing a population of first cDNA strands;
removing the mRNA;
synthesizing a second cDNA strand complementary to each first strand, thereby providing a population of double-stranded cDNA molecules;
digesting the double-stranded cDNA molecules with a Type II or Type IIS restriction enzyme to provide a population of digested double-stranded cDNA molecules, each digested double-stranded cDNA molecule having a cohesive end provided by the restriction enzyme digestion;
ligating a population of cohesive adaptor oligonucleotides to the cohesive end of each of the digested double-stranded cDNA molecules, the cohesive adaptor oligonucleotides each comprising an end sequence complementary to a cohesive end, a first forward primer annealing sequence, and a second forward primer annealing sequence between the first forward primer annealing sequence and the cohesive end, thereby providing double-stranded template cDNA molecules each comprising a first strand and a second strand wherein the first strand of the double-stranded template cDNA molecules each comprise a 3′ terminal cohesive adaptor oligonucleotide and the second strand of the double-stranded template cDNA molecules each comprise a 3′ sequence complementary to the oligot adaptor sequence;
purifying said double-stranded template cDNA molecules;
performing a first polymerase chain reaction on the double-stranded template cDNA molecules having a sequence complementary to a 3′ end of an mRNA using a first forward primer, which comprises a sequence which anneals to the first forward primer annealing sequence, and a first back primer, which comprises a sequence which anneals to the first back primer annealing sequence;
performing a second polymerase chain reaction amplification on products of the first polymerase chain reaction using a population of second forward primers and a population of second back primers,
wherein the second forward primers each comprise a sequence which anneals to a second forward primer annealing sequence of a cohesive adaptor oligonucleotide; and
where the restriction enzyme is a Type II enzyme the second forward primers each comprise at least one 3′ terminal variable nucleotide and optionally more than one 3′ terminal variable nucleotides wherein the variable nucleotide is, or at a corresponding position within the variable nucleotides each second forward primer has, a nucleotide selected from A, T, C and G, whereby the population of second forward primers primes synthesis in the polymerase chain reaction of first strand product DNA molecules each of which is complementary to the first strand of a template cDNA molecule that comprises adjacent to the primer annealing sequence within the first strand of the template CDNA molecule a nucleotide or sequence of nucleotides complementary to the variable nucleotide or nucleotides of a second forward primer within the population of second forward primers; or
where the restriction enzyme is a Type IIS enzyme the second forward primers prime synthesis in the polymerase chain reaction of first strand product DNA molecules each of which is complementary to the first strand of a template cDNA molecule that comprises within the first strand of the template cDNA molecule a sequence of nucleotides complementary to an end sequence of a cohesive adaptor oligonucleotide in the population of cohesive adaptor oligonucleotides;
the second back primers comprise an oligoT sequence and a 3′ variable portion conforming to the following formula: (G/C/A) (X)n wherein X is any nucleotide, n is zero, at least one or more than one; whereby the population of second back primers primes synthesis in the polymerase chain reaction of second strand product DNA molecules each of which is complementary to the second strand of a template cDNA molecule that comprises adjacent to polyA within the second strand of the template cDNA molecule a nucleotide or nucleotides complementary to the variable portion of a second back primer within the population of second back primers;
whereby performing the polymerase chain reaction amplifications provides a population of double-stranded product DNA molecules each of which comprises a-first strand product DNA molecule and a second strand product DNA molecule.

2. A method according to claim 1 further comprising separating double-stranded product DNA molecules on the basis of length; and
detecting said double-stranded product DNA molecules;
whereby a pattern for the population of mRNA molecules present in the sample is provided by combination of length of said double-stranded product DNA molecules and (i) second forward primer variable nucleotide or nucleotides, where a Type II restriction enzyme is employed, or (ii) cohesive adaptor oligonucleotide end sequence, where a Type IIS restriction enzyme is employed.

3. A method according to claim 1 or claim 2 that further comprises:
generating an additional pattern for the sample using a second, different Type II or Type IIS restriction enzyme, and comparing the patterns generated using at least two different Type II or Type IIS restriction enzymes in separate experiments with a database of signals determined or predicted for known mRNA's.

4. A method according to claim 3 wherein patterns generated using at least two different Type II or Type IIS restriction enzymes in separate experiments with a database of signals determined or predicted for known mRNA's by:
(i) listing all mRNA's in the database which may correspond to a double-stranded product DNA in each experiment, forming a list of mRNA molecules possibly present in the sample for each experiment, and
(ii) for each experiment listing mRNA's which definitely do not correspond to a double-stranded product DNA molecule, forming a list of mRNA molecules definitely not present in the sample for each experiment, then
(iii) removing the mRNA molecules definitely not present in the sample from the list of mRNA molecules possibly present for each experiment, and
(iv) generating a list of mRNA molecules possibly present in the sample and mRNA molecules definitely not present in the sample by combining each list generated for each experiment in (iii);
thereby providing a profile of mRNA molecules present in the sample.

5. A method according to claim 4 which comprises comparing the patterns generated using at least two different Type II or Type IIS restriction enzymes in separate. experiments with a database of signals determined or predicted for known mRNA's, by:
(i) listing all mRNA's in the database which may correspond to a double-stranded product DNA in each experiment, and forming a set of equations of the form Fi=m₁+m₂+m₃, wherein Fi is the intensity of the signal from the fragment, the numerals are the mRNA identity and wherein each mRNA which may correspond to a double-stranded product DNA appears as a term on the right-hand side;
(ii) for each experiment listing mRNA's which definitely do not correspond to double-stranded product DNA in each experiment, and writing for each gene which definitely does not correspond to a double-stranded product DNA in each experiment an equation of the form 0=m₄, wherein the numeral is the mRNA identity;
(iii) combining the sets of equations to form a system of simultaneous equations wherein the number of equations is greater than the number of genes in the organism;
(iv) determining an estimate of the expression level of each gene by solving.the system of simultaneous equations, thereby providing a profile of mRNA molecules present in the sample.

6. A method according to any one of claims 1 to 5 wherein the following primer sequences are employed:
first forward primer of the following sequence: 5′-AGGACATTTGTGAGTCAGGC-3′ (SEQ ID NO. 26),
first back primer of the following sequence: 5′-TTCACGCTGGACTGTTTCGG-3′ (SEQ ID NO. 27),
second forward primer of the following sequence:
5′-GTGTCTTGGATGC-3′ (SEQ ID NO. 35), and
second back primer of the following sequence:
5′-(T)_{z}VN₁N₂, wherein z is 10-40, V is A, G or C, N₁ is optional and if present is A, G, C or T, and N₂ is optional and if present is A, G, C or T.

7. A method of amplifying cDNA fragments to provide a population of double-stranded product DNA molecules, each cDNA fragment comprising an upper strand that comprises a copy of a 3′ fragment of an mRNA molecule comprising a polyA tail, and a lower strand that is complementary to the upper strand, wherein the upper strand comprises at its 5′ terminus the following adaptor (1) sequence:
5′-AGGACATTTGTGAGTCAGGCGTGTCTTGGATGC-3′, and the lower strand comprises at its 3′ terminus the following adaptor (2) sequence:
5′-p(N)ₓGCATCCAAGACACGCCTGACTCACAAATGTCCT-3′, and wherein the lower strand comprises at its 5′ terminus the following adaptor (3) sequence:
5′-CCAATTCACGCTGGACTGTTTCGG-(T)_{y}-3′ and the upper strand comprises at its 3′ terminus the following adaptor (4) sequence:
5′-(A)_{y}-CCGAAACAGTCCAGCGTGAATTGG-3′,
wherein the upper and lower strands.are provided by ligation of adaptors of adaptor sequence (1) and (2) following restriction digest of cDNA fragments, wherein N is A, T, C or G, and wherein x corresponds to the number of bases of overhang created by the restriction digest;
the method comprising performing nested polymerase chain reaction,
wherein a first polymerase chain reaction is performed with a first forward primer of the following sequence:
5′-AGGACATTTGTGAGTCAGGC-3′ (SEQ ID NO. 26), and a first back primer of the following sequence:
5′-TTCACGCTGGACTGTTTCGG-3′ (SEQ ID NO. 27), and
wherein a second polymerase chain reaction is performed with a second forward primer of the following sequence:
5′-GTGTCTTGGATGC-3′ (SEQ ID NO. 35), and a second back primer of the following sequence:
5′-(T)_{z}VN₁N₂, wherein z is 10-40, V is A, G or C, N₁ is optional and if present is A, G, C or T, and N₂ is optional and if present is A, G, C or T.

8. A method according to any one of claims 1 to 7 wherein the second back primers are labelled.

9. A method according to claim 8 wherein the second back primers are labelled with fluorescent dyes readable by a sequencing machine.

10. A method according to any one of claims 1 to 9 comprising determining the length of double-stranded product DNA molecules in the population by electrophoresis and comparison with a size standard that comprises tandemly ligated oligonucleotides of the following sequences:
(SEQ ID NO. 28)
5′-CTAGTCCTGCAGGTTTAAACGAATTCGCCCTTGGATGCCT-3′,
and
(SEQ ID NO. 29)
3′-AGGACGTCCAAATTTGCTTAAGCGGGAACCTACGGAGATC-5′.

11. A method according to any one of claims 1 to 10 comprising determining length of double-stranded product DNA molecules in the population by electrophoresis and employing an internal control polynucleotide of the sequence:
(SEQ ID NO. 30)
5′-AGGACATTTGTGAGTCAGGCGTGTCTTGGATGC(N)ₚV(A)_{z′}ACCG
AAACAGTCCAGCGTGAATTGG-3′
wherein N is any nucleotide (A, T, C or G) and p is a number to provide a desired overall length of polynucleotide, wherein p is preferably 600-700, V′ is T, C or G, and z′ is 10-40.

12. A set of primers for nested polymerase chain reaction to amplify cDNA copies of mRNA fragments comprising polyA tails, wherein the set comprises
a first forward primer of the following sequence: 5′-AGGACATTTGTGAGTCAGGC-3′ (SEQ ID NO. 26),
a first back primer of the following sequence: 5′-TTCACGCTGGACTGTTTCGG-3′ (SEQ ID NO. 27),
a second forward primer of the following sequence: 5′-GTGTCTTGGATGC-3′ (SEQ ID NO. 35), and
a second back primer of the following sequence:
5′-(T)_{z}VN₁N₂, wherein z is 10 to 40, V is A, G or C,
N₁ is optional and if present is A, G, C or T, and N₂ is optional and if present is A, G, C or T.

13. A kit comprising:
a set of primers according to claim 12; and
a set of adaptor oligonucleotides of the following sequences:
wherein a first adaptor oligonucleotide has an upper strand sequence:
5′-AGGACATTTGTGAGTCAGGCGTGTCTTGGATGC-3′ (SEQ ID NO. 31), and a lower strand sequence:
5′-p(N)ₓGCATCCAAGACACGCCTGACTCACAAATGTCCT-3′, and wherein a second adaptor oligonucleotide has lower strand sequence:
5′-CCAATTCACGCTGGACTGTTTCGG-(T)_{y}-3′ and an upper strand sequence:
5′-(A)_{y}-CCGAAACAGTCCAGCGTGAATTGG-3′;
wherein N is A, T, C or G, and wherein x is 1, 2, 3 or 4.

14. A kit according to claim 13 comprising a size standard that comprises tandemly ligated oligonucleotides of the following sequences:
(SEQ ID NO. 28)
5′-CTAGTCCTGCAGGTTTAAACGAATTCGCCCTTGGATGCCT-3′,
and
(SEQ ID NO. 29)
3′-AGGACGTCCAAATTTGCTTAAGCGGGAACCTACGGAGATC-5′;
wherein the tandemly ligated oligonucleotides are amplifiable from vectors wherein the tandemly ligated oligonucleotides are inserted between an upstream primer binding site and a downstream oligoA sequence.

15. A kit according to claim 14 which comprises a population of vectors, wherein vectors in the population comprise tandemly ligated oligonucleotides of between 0 and 25 repeats, amplification using said a primer that binds said upstream primer binding site and a primer that binds said oligoA providing a population of size marker oligonucleotides of different lengths.

16. A kit according to any one of claims 13 to 15 comprising an internal control polynucleotide of the sequence:
(SEQ ID NO. 30)
5′-AGGACATTTGTGAGTCAGGCGTGTCTTGGATGC(N)ₚV(A)_{z′}ACCG
AAACAGTCCAGCGTGAATTGG-3′
wherein N is any nucleotide (A, T, C or G) and p is a number to provide a desired overall length of polynucleotide, wherein p is preferably 600-700, V′ is T, C or G, and z′ is 10-40.

17. A kit according to any one of claims 13 to 16 comprising one or more Type II or Type IIS restriction enzymes.

18. A kit according to any one of claims 13 to 17 comprising components for use in performance of a polymerase chain reaction.
